SEQUENCE LISTING (14 sequences)

SEQ ID NO: 1 - DNA, 18 bp, Artificial Sequence
  Description of Artificial Sequence forward primer sequence for amplifying Mycoplasma and Acholeplasma 16S rDNA sequences
gacagatggtgcatggtt

SEQ ID NO: 2 - DNA, 22 bp, Artificial Sequence
  Description of Artificial Sequence reverse primer sequence for amplifying Mycoplasma and Acholeplasma 16S rDNA sequences
catattgcttctctttgtaccg

SEQ ID NO: 3 - DNA, 18 bp, Acholeplasma laidlawii
tacaggtggtgcacggtt

SEQ ID NO: 4 - DNA, 22 bp, Acholeplasma laidlawii
cctatcgcttctctttgttcca

SEQ ID NO: 5 - DNA, 22 bp, Mycoplasma pirum
ggattcgcaactgtttgtaatg

SEQ ID NO: 6 - DNA, 20 bp, Homo sapiens
atgggtcagaaggattccta

SEQ ID NO: 7 - DNA, 24 bp, Mus musculus
tggcagaactgttccctcatcttc

SEQ ID NO: 8 - DNA, 17 bp, Homo sapiens
tccatgtcgtcccagtt

SEQ ID NO: 9 - DNA, 18 bp, Mycoplasma salivarium
  variation (18): N at position 18 indicates any nucleotide base
gacagatggtgcatggtn

SEQ ID NO: 10 - DNA, 18 bp, Mycoplasma pirum
gacaggtggtgcatggtt

SEQ ID NO: 11 - DNA, 22 bp, Mycoplasma fermentans
cacttcgcttctctttgtaccg

SEQ ID NO: 12 - DNA, 24 bp, Mus musculus
gggcgatgatgttgatgacgtaga

SEQ ID NO: 13 - DNA, 18 bp, E. coli
gaaaggcgcgcgatacag

SEQ ID NO: 14 - DNA, 19 bp, E. coli
gtcccgccctactcatcga

(19) United States
(12) Patent Application Publication
(10) Pub. No.: US 2005/0250112 A1
(43) Pub. Date: Nov. 10, 2005

(54) Nucleic acids, compositions, methods, and kits for detecting mycoplasma and acholeplasma species

(76) Inventors: Gothami A. Padmabandu, San Diego, CA (US); Fatemeh Salehi, San Diego, CA (US); Peter Louis Pingerelli, Escondido, CA (US); Reinhold Dietrich Mueller, San Diego, CA (US)

Correspondence Address: MOAZZAM LATIMER LLP, 1474 NORTH POINT VILLAGE CENTER #320, RESTON, VA, 20194-1190, US

(21) Appl. No.: 10/840,672

(22) Filed: May 7, 2004

(51) Int. Cl. C12Q 1/68; C07H 21/04

(52) U.S. Cl. 435/6; 536/24.1

(57) ABSTRACT

The invention relates to nucleic acids, methods, compositions, and kits for the PCR-based detection of Mycoplasma and Acholeplasma bacterial species. The nucleic acids, methods, compositions, and kits provide for increased specificity and sensitivity of PCR-based Mycoplasma bacterial assays. Primer sets and PCR-based assays are provided that amplify and detect conserved 16S rRNA gene sequences from multiple Mycoplasma and Acholeplasma species while avoiding amplification and detection of non-target sequences.

### FIELD OF THE INVENTION

The invention relates to the detection of bacterial species in biological samples. In particular, the invention relates to detection of nucleic acids specific for Mycoplasma and Acholeplasma species.

### BACKGROUND OF THE INVENTION

Various species of Mycoplasma and Acholeplasma are involved in human and animal pathologies. Although the first Mycoplasma species was identified in association with bovine pleuropneumonia, it has since been identified as the causative agent of lung disease in humans. Likewise, Acholeplasma species have been implicated in waterfowl, swine, cattle, and human disease.

Mycoplasma contamination of eukaryotic cell cultures is also a common problem, leading to unreliable experimental results and possibly unsafe biological products. Contamination is typically due to presence of Mycoplasma in the original cells used for culture, cross-contamination of laboratory stocks, contamination from compositions added to cell cultures during maintenance or experimental procedures, or transfer from infected laboratory personnel. Although it is widely accepted that ultraviolet and gamma irradiation kills Mycoplasma, these small bacteria pass easily through commonly used 0.22-micron sterilization filters. In addition, certain antibiotics are unsuitable for maintaining a Mycoplasma-free culture because of the lack of a Mycoplasma cell wall. Some studies suggest that the prevalence of Mycoplasma contamination in cell cultures is as high as 15% (DelGuidice and Hopps, 1978; Barile and Razin, 1979; McGarrity and Kotani, 1985). Such contamination can adversely affect experiments by altering eukaryotic cell surface antigens, chromosomal structure, metabolic rates, protein expression patterns, and transfection efficiency.

Detection of Mycoplasma and Acholeplasma in cultured cells and tissues is thus critical for the reliability and reproducibility of experimental data. traditional methods of detection are difficult and time consuming, due to the fastidiousness and slow growth of Mycoplasma and Acholeplasma species in culture (Barile and Razin, 1979). Mycoplasma and Acholeplasma culture tests require 15-30 days and the interpretation of the data requires a trained eye. and, while staining with 4′,6′-diamidino-2-phenylindole hydrochloride (DAPI) or Hoechst stain reduces turn-around time compared to the culture method, the results can still be difficult to interpret. Immunofluorescence detection is also subjective and insensitive, particularly for Acholeplasma (Tang et al., 1999).

A number of specific Mycoplasma and/or Acholeplasma detection assays for detection in both clinical and cell culture settings have been described, for example by: Harasawa et al., Res. Microbiol. 144:489-493, 1993; Blazek et al., J. Immunol. Meth. 131:203-212, 1990; Hopert et al., J. Immunol. Meth. 164:91-100, 1993; McGarrity et al., In Vitro Cell. Dev. Biol. 22:301-304, 1986; Uphoffet al., Leukemia 6:335-341, 1992; van Kuppeveld, Appl. Environ. Microbiol. 58:2606-2615, 1992; van Kuppeveld, Appl. Environ. Microbiol. 60:149-152, 1994; Wirth et al., Cytotechnology 16:67-77, 1994; Corless et al., J. Clin. Microbiol. 38:1747-1752, 2000; Kong et al., Appl. Environ. Microbiol. 67:3195-3200, 2001; Yoshida et al., J. Clin. Microbiol. 40:1451-1455, 2002; Loens et al., J. Clin. Microbiol. 40:339-1345, 2002; and Eastick et al., J. Clin. Pathol.: Mol. Pathol. 56:25-28, 2003.

In addition, Uphoff et al., Leukemia 16:289-293, 2002, describes an assay using a mixture of 9 different oligonucleotide primers that amplify 16S rRNA genes from M. arginini, M. fermentans, M. hominis, M. hyorhinis, M. orale, and A. laidlawii. Further, Dussurget and Roulland-Dussoix, Appl. Environ. Microbiol. 60:953-959, 1994, describes the use of a mixture of polymerase chain reaction (PCR) primers that amplify 16S rRNA gene sequences to detect M. arginini, A. laidlawii, M. hyorhinis, M. orale, and M. fermentans.

Some kits to detect Mycoplasma are commercially available. For example, the Mycoplasma Plus™ PCR Primer Set kit (Cat. # 302008, Stratagene, La Jolla, Calif.) uses PCR and restriction fragment analysis to detect the presence, and identify the particular species, of Mycoplasma or Acholeplasma in a sample. Likewise, the MycoSensor™ PCR Assay Kit (Cat. # 302108, Stratagene, La Jolla, Calif.) is a gel-based PCR assay for the detection of Mycoplasma and Acholeplasma species. The ATCC Mycoplasma detection kit (Cat. # 90-1001K, American Type Culture Collection, Manassas, Va.) uses a nested PCR process to amplify the conserved region between 16S and 23S rRNA. In addition, a PCR Mycoplasma detection set from Takara Bio Inc. (Otsu, Shiga, Japan) amplifies the conserved region between 16S and 23S rRNA. The MycoTect™ kit (Cat. No. 15672-017, Gibco/Invitrogen, Carlsbad, Calif.) is also available, and detects Mycoplasma directly in cell culture using 6-MPDR.

In addition to these kits, a real-time Mycoplasma detection kit is commercially available. The VenorGeM-QP® from Minerva Biolabs (Berlin, Germany) targets the 16S rDNA of Mycoplasma, and utilizes two probes for detection, each with a different fluorescent dye. According to the manufacturer, the kit can detect as few as 30 copies of a Mycoplasma genome. The protocol provided by the manufacturer states that 45 cycles of amplification should be used. However, the manufacturer does not state whether 45 amplification cycles is sufficient to detect as few as 30 Mycoplasma genomes.

Furthermore, while it is known to use SYBR® (Molecular Probes, Eugene, Oreg.) Green for quantitative PCR (QPCR) applications (e.g., Brilliant® SYBR® Green QPCR and QRT-PCR products from Stratagene (La Jolla, Calif.) under product numbers 600546, 600548, 600552, and others); the DyNAmo™ HS SYBR® Green QPCR kit from Finnzymes (Espoo, Finland); Platinum® SYBR® Green qPCR SuperMix UGD (Invitrogen, Carlsbad, Calif.); SYBR® Green JumpStart Taq ReadyMix (Sigma, St. Louis, Mo.); SYBR® Green QPCR Master Mix (Applied Biosystems, Foster City, Calif.); and the QuantiTect™ SYBR® Green PCR and RT-PCR kit provided by QIAGEN (Valencia, Calif.)), none of these products are described as having particular advantages with respect to Mycoplasma or Acholeplasma detection.

While useful for detection of various nucleic acids and bacteria, some of the methods, kits, and systems discussed above have at least one limitation, which, if overcome, would improve its usefulness. Others are not disclosed as useful in QPCR assays, and thus might be inapplicable for certain needs.

### SUMMARY OF THE INVENTION

The present invention encompasses nucleic acids, methods, compositions, and kits for sensitive and specific detection of Mycoplasma and Acholeplasma species in samples. The invention utilizes specific primers and amplification methods that permit real-time monitoring of amplification products during QPCR reactions. Real-time monitoring according to the invention can be used to ensure that specific amplification of target nucleic acids is occurring, and to differentiate between QPCR signals due to specific amplification of target nucleic acids and possible amplification of contaminating or otherwise non-target nucleic acids. In embodiments, the invention utilizes the differing melting temperatures (Tm) of various potential QPCR products to identify whether they are specific target amplification products, non-specific, non-target amplification products, specific positive control products, or primer-dimer products.

In a first aspect, the invention provides nucleic acids that can be used in detecting Mycoplasma species, Acholeplasma species, or combinations of one or more species from these two genera. The nucleic acids can be oligonucleotides that can function as primers for acellular amplification reactions. The nucleic acids can also be genomic or sub-genomic nucleic acids that can be used as controls for monitoring the progression, specificity, and/or sensitivity of the methods of the invention.

In a second aspect, the invention provides methods of acellular amplification of target nucleic acids. The methods of the invention use at least two oligonucleotides to specifically amplify Mycoplasma and/or Acholeplasma nucleic acids, while essentially avoiding amplification of nucleic acids from other bacteria or eukaryotes. In general, the methods comprise providing purified target nucleic acids, and amplifying and detecting target sequences within the target nucleic acids.

In a third aspect, the invention provides compositions. In general, the compositions comprise at least two oligonucleotide primers that can be used to specifically amplify Mycoplasma and/or Acholeplasma nucleic acids. The compositions can also contain reagents, solvents, and other nucleic acids for practicing the methods of the invention.

In a fourth aspect, the invention provides kits for detecting Mycoplasma and/or Acholeplasma species in a sample. The kits can comprise, in one or more packaged combinations, two or more oligonucleotide primers, reagents for performing the methods of the invention, solvents for performing the methods of the invention, and nucleic acid templates for use as positive controls or specificity controls.

### BRIEF DESCRIPTION OF THE DRAWINGS

FIG. 1 shows a schematic diagram of one embodiment of the method of the invention, in which Mycoplasma and/or Acholeplasma species are detected in real-time using SYBR® Green I.

FIG. 2A shows a diagram of the primers used for amplifying the conserved region of the 16S rRNA gene according to one embodiment of the invention.

FIG. 2B shows a diagram of the primers used for amplifying the Amplification Control (AC) template according to one embodiment of the invention.

FIG. 3 shows the results of experiments demonstrating that the Mycoplasma 16S rRNA primer set disclosed herein detects 10⁶ copies of genomic DNA from eight Mycoplasma/Acholeplasma species of interest according to one embodiment of the invention. The amplification plot was generated on a Stratagene MX3000P instrument. Displayed is the semi-log view. Amplification was performed using the Brilliant® SYBR® Green Master Mix (Stratagene), the 16S rRNA primer set Mycoplasma spp. F, M. spp. R2, A. laidlawii F, A. laidlawii R2, M. pirum R2 (SEQ ID NOs:1-5, respectively; Table 1), and 10⁶ copies of genomic DNA from each of the eight species indicated as the most common cell culture contaminants (Tang et al., 1999), or water as a no-template control. Note that curves for the no-template control do not show in this amplification plot because the fluorescence was below 0.01.

FIG. 4A is a dissociation curve of the same samples shown in FIG. 3. It shows determination of the melting point of PCR products resulting from the methods according to embodiments of the invention. The fist derivative of the SYBR® Green melting curve is shown in the figure.

FIG. 4B lists the species used to generate the dissociation curve of FIG. 4A and their respective melting temperatures.

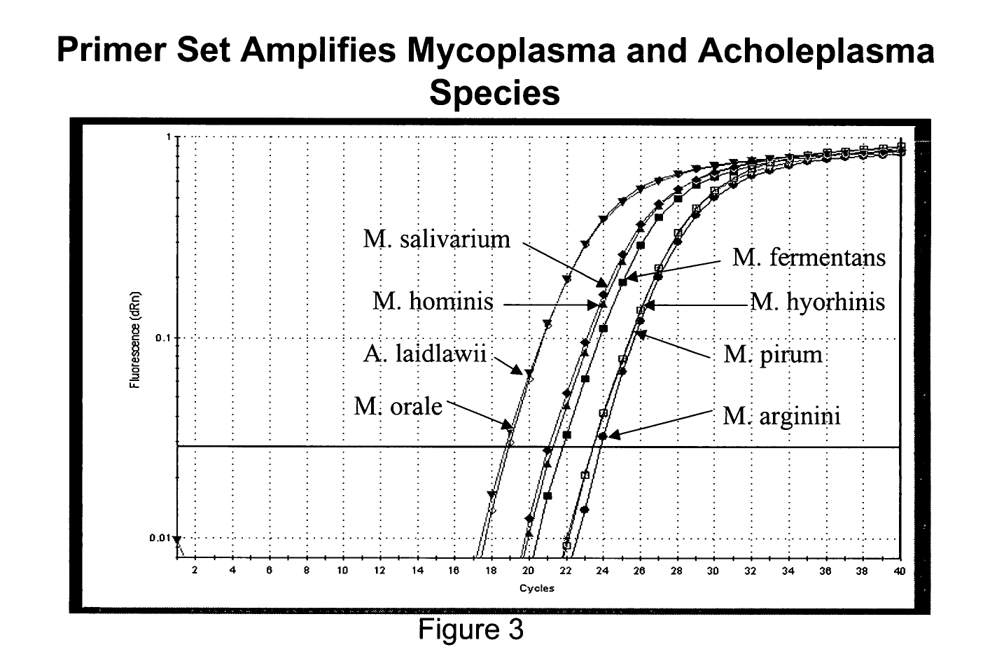

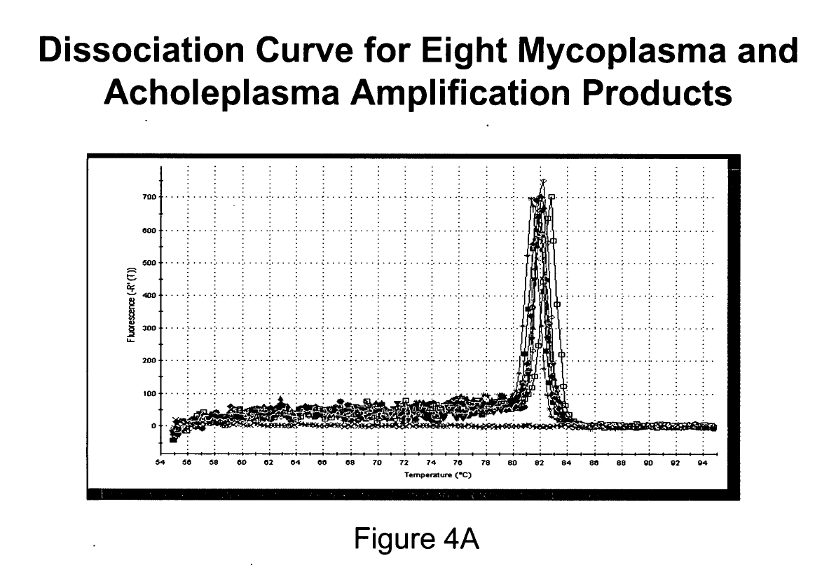

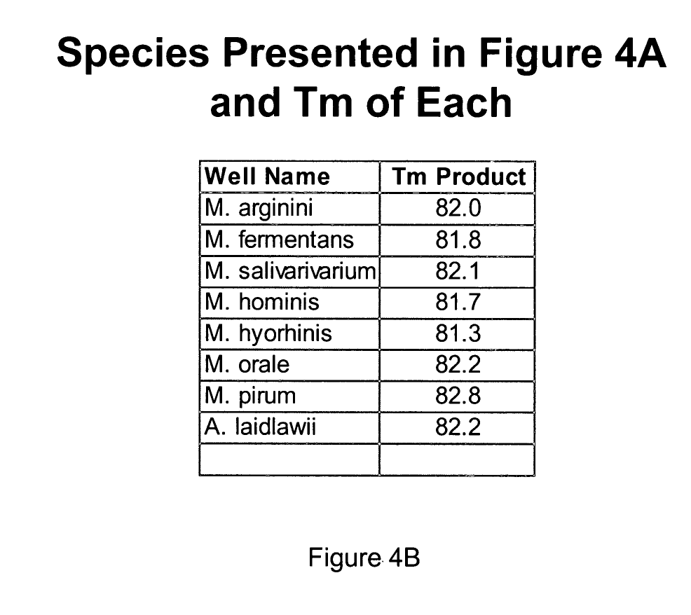

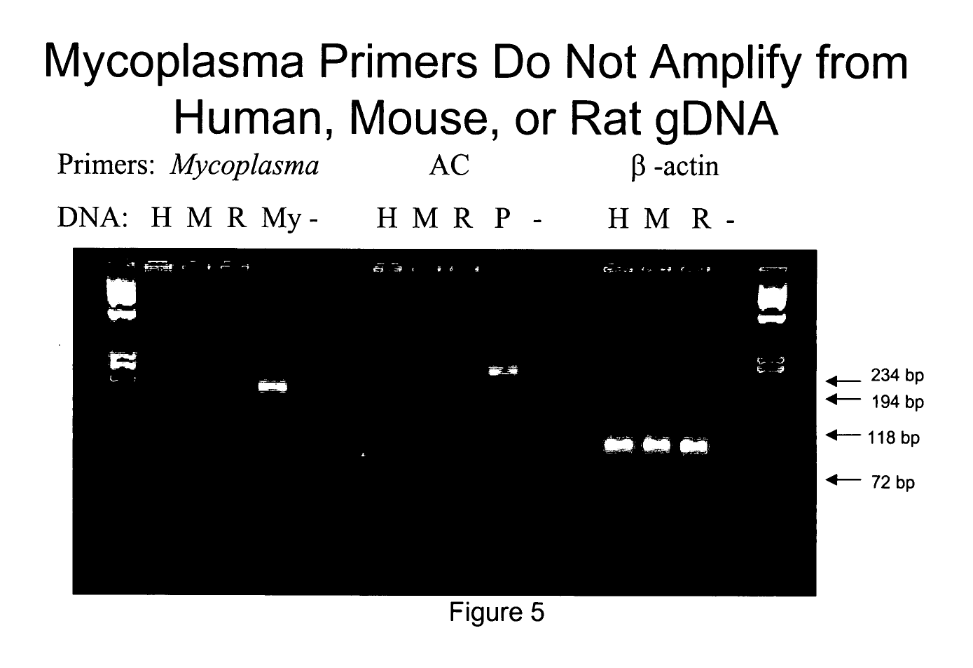

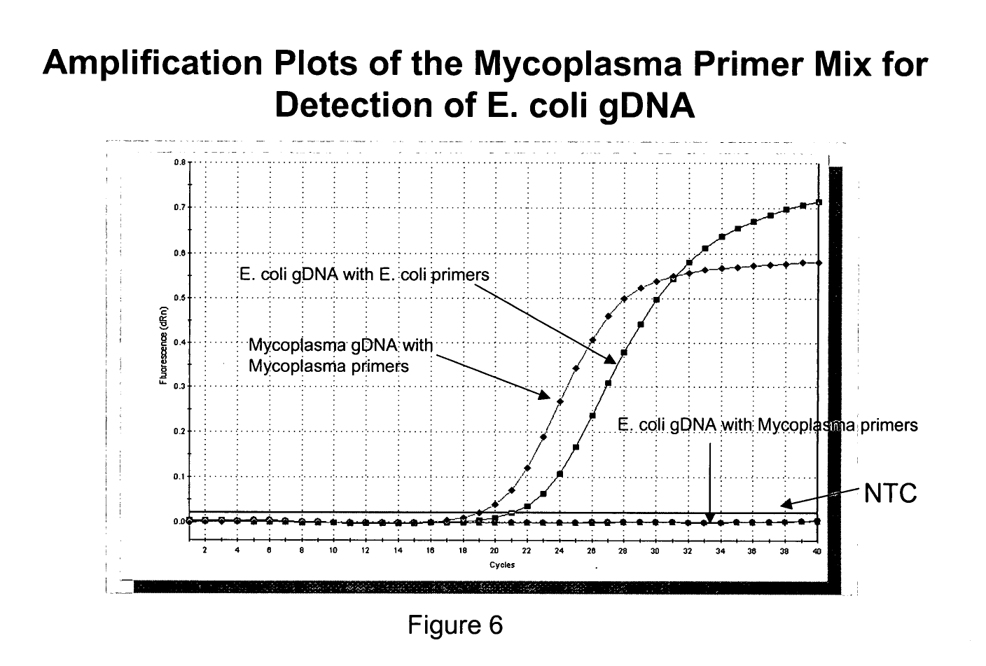

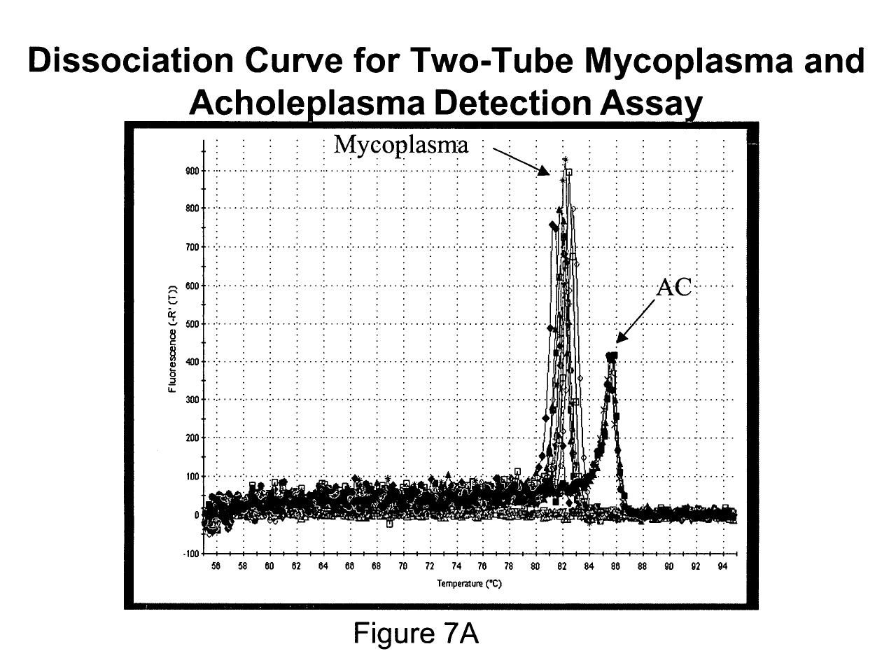

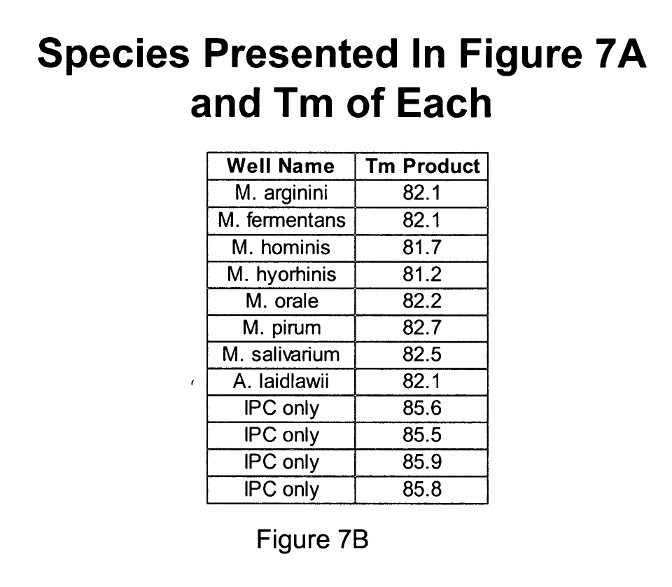

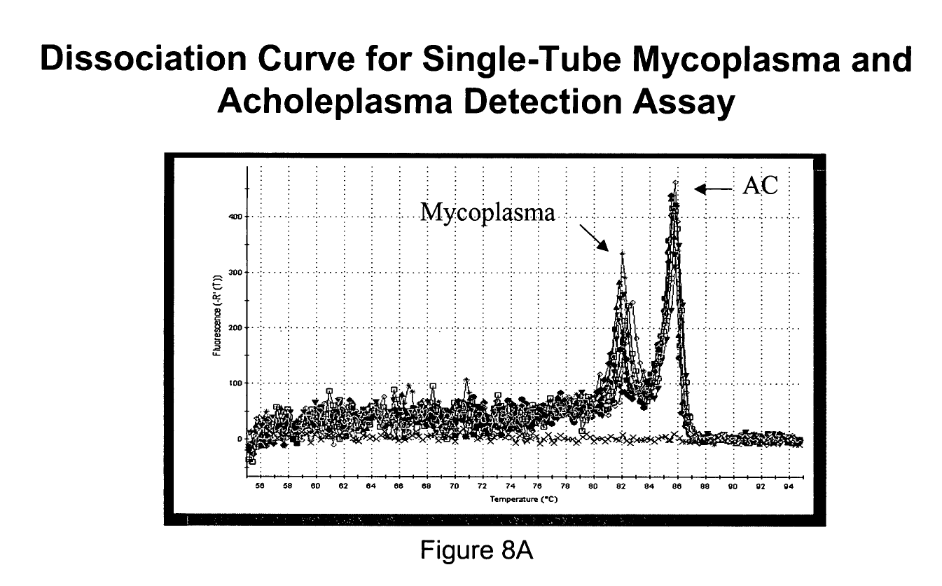

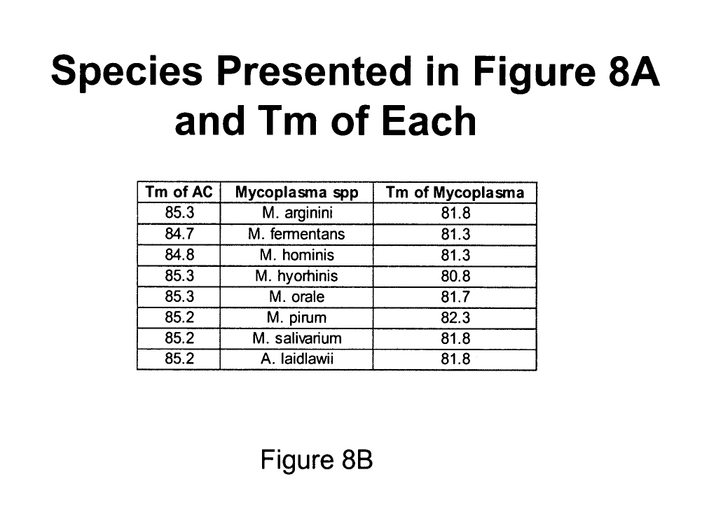

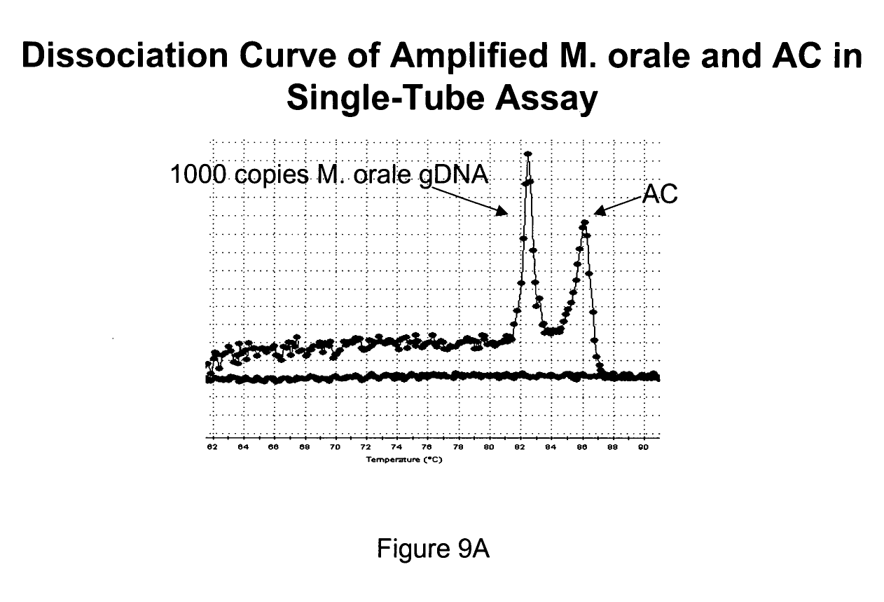

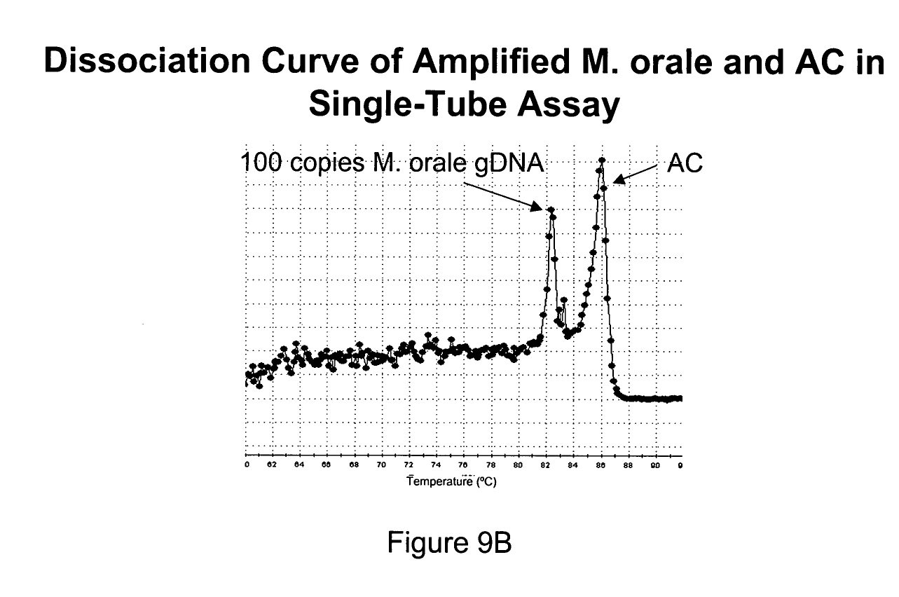

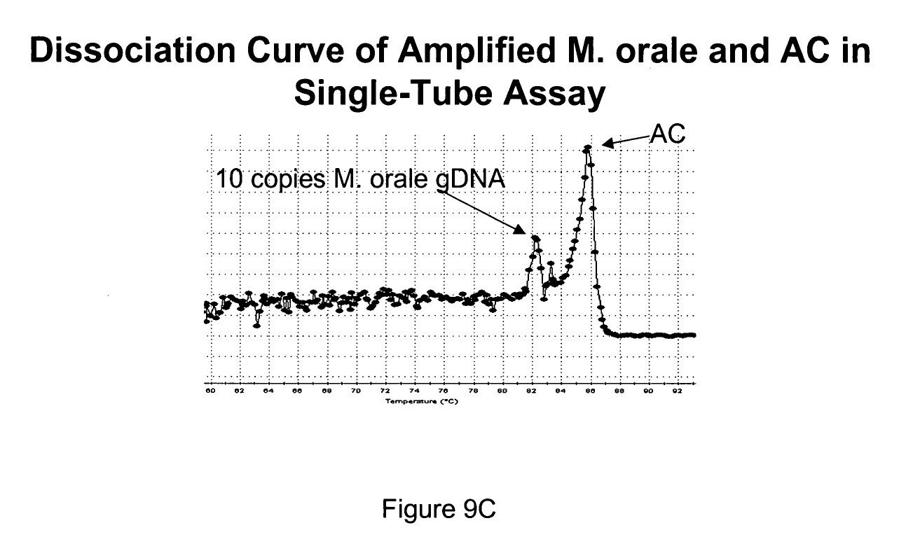

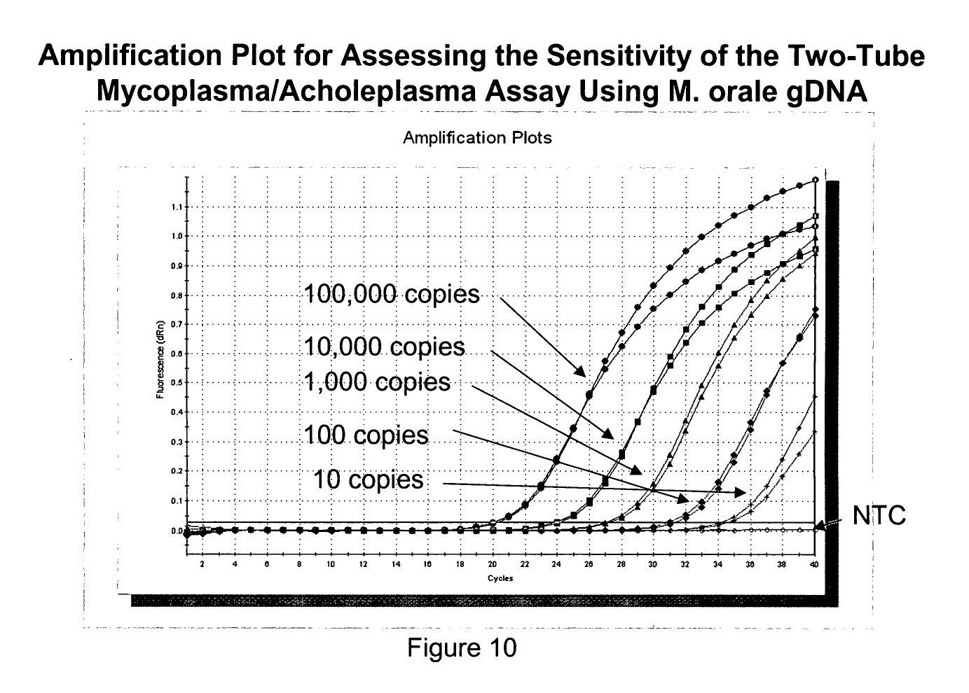

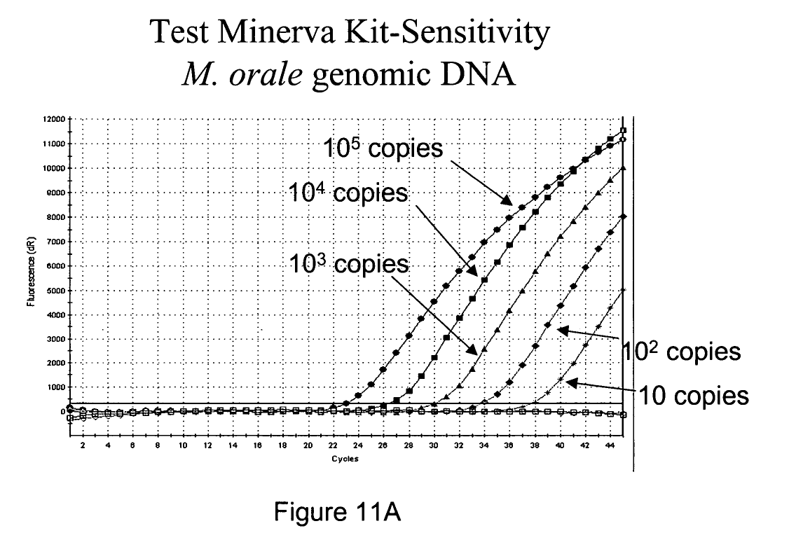

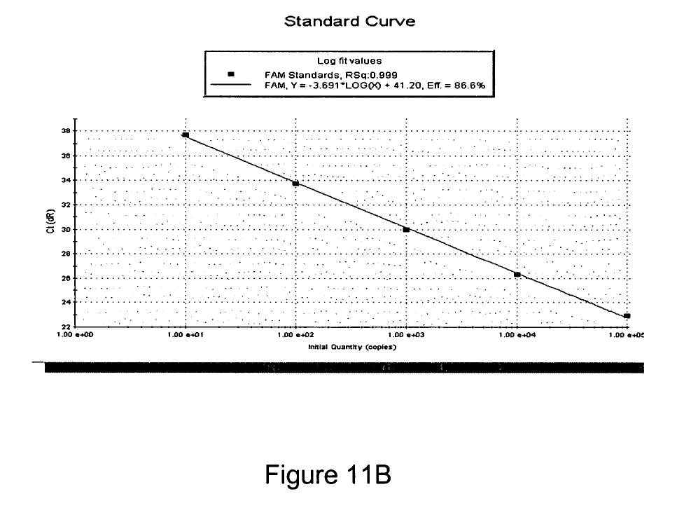

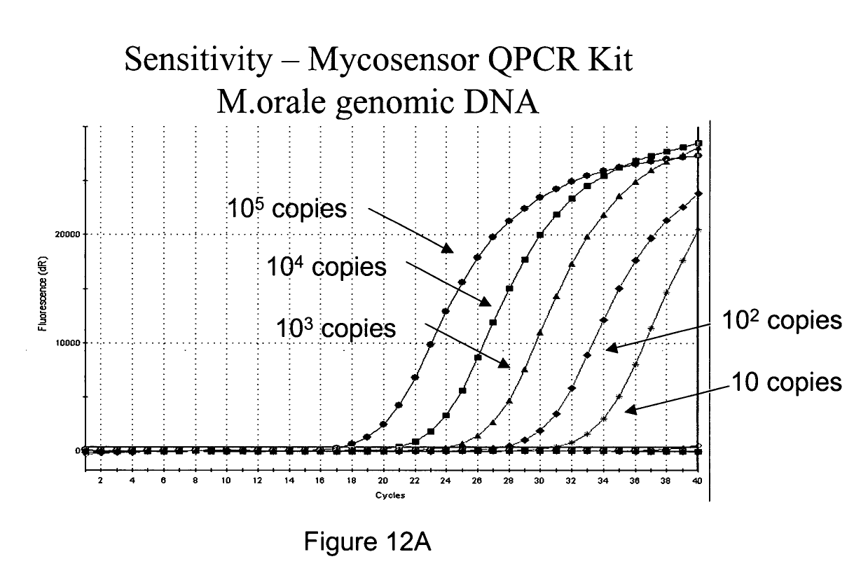

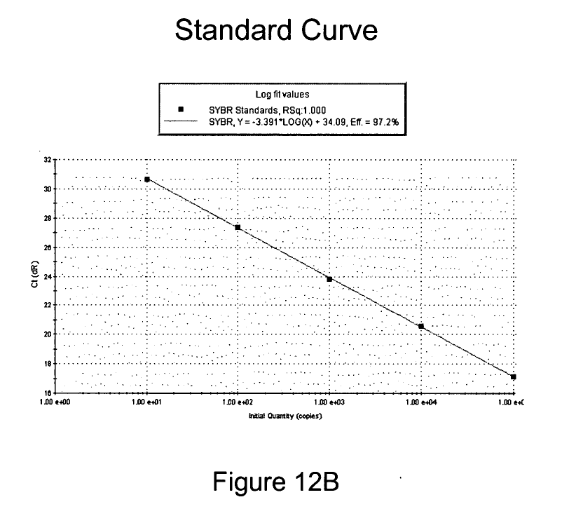

10⁶ genomic copies of each of the Mycoplasma/Acholeplasma species listed in FIG. 4B were amplified in a real-time thermocycler (as described in the description of FIG. 3). After completion of QPCR, the amplified DNA in the samples was melted and allowed to anneal while the temperature was adjusted to a low level (e.g., 55° C.). When the temperature of the samples was slowly ramped up to about 95° C., the annealed DNA strands resulting from the PCR reaction melted and thereby lost the ability to bind SYBR® Green. The temperature at which the fluorescence of SYBR® Green significantly dropped defined the melting point of the DNA in each sample.

FIG. 5 shows the results of experiments examining whether the Mycoplasma 16S rRNA primer set M. spp. F, M. spp. R2, A. laidlawii F, A. laidlawii R2, M. pirum R2 (SEQ ID NO:1-5, respectively; Table 1) amplifies a product from human, mouse, or rat genomic DNA templates according to one embodiment of the invention. The figure shows that the primers are specific for Mycoplasma 16S rRNA sequences.

Legend: Primers−“Mycoplasma”=Mycoplasma primers, “AC”=amplification control primers, β-actin=primers specific for human/mouse/rat β-actin; DNA−“H”=human genomic DNA, “M”=mouse genomic DNA, “R”=rat genomic DNA, “My”=Mycoplasma orale genomic DNA, “−”=no DNA added to reaction mix, “P”=plasmid pBMG419 DNA, containing an amplification control, mouse muscle nicotinic acetylcholine receptor, gamma subunit DNA; molecular weight markers (ΦX 174 HaeIII ladder) are shown in the far left and far right lanes).

FIG. 6 shows a real-time analysis of the Mycoplasma primer set of the invention (as described above) for species specificity. The primer set was added to Brilliant® SYBR® Green Master Mix. Mycoplasma orale gDNA, E. coli gDNA, or only water (NTC) was added to the reactions, which were then amplified. The amplification plot of the figure shows that only Mycoplasma DNA generated a signal. This shows that the primer set is specific for Mycoplasma. One nanogram of E. coli gDNA was amplified with Mycoplasma primers according to the invention or primers specific for the E. coli 23S rDNA. The figure shows that no E. coli gDNA was detected with the Mycoplasma primers of the invention, whereas the positive control for amplification (E. coli gDNA with E. coli-specific primers) showed amplification.

FIG. 7A shows a dissociation curve for a Mycoplasma detection assay in the 2-tube format according to the invention, where Mycoplasma/Acholeplasma nucleic acids and an amplification control (AC) are detected.

FIG. 7B lists the species or nucleic acids used to generate the dissociation curve of FIG. 7A.

FIG. 8A shows a dissociation curve for a Mycoplasma detection assay in the single-tube format according to the invention.

FIG. 8B lists the species or nucleic acids used to generate the dissociation curve of FIG. 8A.

10⁵ copies of genomic DNA from the listed species, each including 1,000 copies of amplification control, were amplified. The data from the melting profile are shown in FIG. 8A. The Tm of the eight bacterial species is about 82° C., as expected. The Tm of the AC in the same tube is about 85° C., and thus can be differentiated from the target template products.

FIG. 9A shows a dissociation curve for Mycoplasma orale detection in the single-tube format of the invention. One thousand copies of M. orale DNA were added to Brilliant® SYBR® Green Master Mix, with 1,000 copies of amplification control included in the same tube.

FIG. 9B shows a dissociation curve for Mycoplasma orale detection in the single-tube format of the invention. One hundred copies of M. orale DNA were added to Brilliant® SYBR® Green Master Mix, with 1,000 copies of amplification control included in the same tube.

FIG. 9C shows a dissociation curve for Mycoplasma orale detection in the single-tube format of the invention. Ten copies of M. orale DNA were added to Brilliant® SYBR® Green Master Mix, with 1,000 copies of amplification control included in the same tube.

FIG. 10 shows an amplification plot of various Mycoplasma orale DNA concentrations. Zero (NTC), 10, 100, 1,000, 10,000, and 100,000 copies of M. orale gDNA were amplified in the presence of HeLa cell culture supernatant in a real-time reader (MX300P instrument from Stratagene). The amplification results show a good linear range for detection and sensitivity of detection from 100,000 copies down to 10 copies of M. orale gDNA in the presence of HeLa cell culture supernatant.

FIG. 11A shows an amplification plot depicting the sensitivity of the Minerva Biolabs VenorGeM® QP real-time Mycoplasma detection kit for Mycoplasma orale detection (10-10⁵ copies gDNA). The kit was used exactly according to the manufacturer's instructions.

FIG. 11B shows a standard curve, as generated on the MX3000P (Stratagene), created from the data presented in FIG. 11A. Based on the standard curve, the efficiency of amplification was 86.6%.

FIG. 12A shows an amplification plot depicting the sensitivity of the methods of the present invention for Mycoplasma orale detection using the primer set of the present invention, using the same starting concentrations of 10-10⁵ of gDNA used in the experiments depicted in FIG. 11.

FIG. 12B shows a standard curve, as generated on the MX3000P (Stratagene), created from the data presented in FIG. 12A. Based on the standard curve, the efficiency of amplification was 97.2%.

### DEFINITIONS

Before proceeding to describe in detail embodiments of the invention, it will be helpful to define certain terms used throughout this document:

As used herein, the phrase “increasing the specificity” of an assay means reducing the frequency or likelihood of false positive assay results. The specificity of an assay is “increased” relative to another assay if there are at least 10% fewer false positive assay results, and preferably at least 20%, 30%, 50%, 75%, 90% or more, up to and including 100% fewer (no false positives) in that assay relative to the other.

One common source of false positive results in bacterial detection methods based on nucleic acid amplification (e.g., PCR-based assays) is nucleic acid from a recombinant host in recombinant polymerase preparations.

As used herein, the term “PCR-based bacterial assay” refers to an assay method for the detection or quantitation of a given bacterial genus or species in a sample, in which the assay comprises PCR amplification with two or more primers that amplify one or more nucleic acid sequences from the targeted bacterial genus or species. A “PCR-based bacterial assay” as the term is used herein is not intended or designed to detect the presence or amount of E. coli bacteria in a sample.

As used herein, the term “aligning” when used in reference to nucleic acid sequences means arranging one or more sequences relative to another such that the greatest number of identical nucleotides are aligned with each other. BCM Search Launcher (via hypertext transfer protocol at //searchlauncher.bcm.tmc.edu/), formatted with BOXSHADE 3.2.1 on the Swiss EMBnet node server (available via hypertext transfer protocol on the world wide web at ch.embnet.org/software/BOX_form.html) can be used for primer sequence alignments. Multiple sequence alignments can also be performed using the BLAST suite of programs available from the NCBI website (see below).

As used herein, the term “homologous” means evolutionarily related, and can be inferred from nucleic acid identity between two sequences. A host bacterial nucleic acid sequence (e.g., an E. coli nucleic acid sequence) is “homologous” to a bacterial target nucleic acid sequence (or vice versa) if it is at least 50% identical to the bacterial target sequence. For the purposes of the present invention, a homologous nucleic acid sequence of a recombinant host bacterium (e.g., E. coli) includes the known sequence or sequences in the recombinant host's genome which has (have) the highest homology to a selected target gene sequence in a target bacterial species. In many instances, homology will be well known, for example, the 16S rRNA gene sequences of Mycoplasma sp. are well known to be homologous to the 16S rRNA gene sequence from E. coli (e.g., the M. orale 16S rRNA gene sequence is a known homolog and is 81% identical to the E. coli 16S rRNA gene sequence). Homology between a target species nucleic acid sequence and a recombinant host (e.g., E. coli) nucleic acid sequence is determined by sequence alignment using, for example, Basic BLAST (e.g., Version 2.0, Altschul et al., Nucleic Acids Res. 25:3389-3402, 1997) set with default parameters (descriptions default=500; alignments default=100; expect=10; filter=off; matrix=BLOSUM62). When two known sequences are to be aligned, the “Blast 2 Sequences” program can be used to align and determine homology (bl2seq; Tatusova & Madden, FEMS Microbiol. Lett. 174:247-250, 1999). The “Blast 2 Sequences” program, available through the NCBI website can be used with default alignment parameters. This program produces the alignment of two given sequences using the BLAST engine for local alignment. Default parameters (for use with the BLASTN program only) are as follows: Reward for a match: 1; Penalty for a mismatch: −2; Strand option Both strands; open gap penalty 5; extension gap penalty 2; gap x_dropoff 50; expect 10.0; word size 11; and Filter (checked).

As used herein, the term “hybridizes,” when used in reference to an oligonucleotide primer, refers to the formation of a hydrogen-bonded base paired duplex with a nucleic acid having a sequence sufficiently complementary to that of the oligonucleotide primer to permit the formation of such a duplex under the conditions used. As the term is used herein, exact complementarity between an oligonucleotide primer and a nucleic acid sequence is not required, with mismatches permitted as long as the resulting duplex is a substrate for extension by a template-dependent nucleic acid extending enzyme. A nucleic acid sequence is “sufficiently complementary” to an oligonucleotide primer if the primer can form a duplex with a molecule comprising the nucleic acid sequence at 55° C. that can be extended by at least one nucleotide by a template-dependent nucleic acid extending enzyme, e.g., a polymerase, in a solution comprising 10 mM Tris-HCl, pH 8.8, 50 mM KCl, 2.0 mM MgCl₂, and 200 μM each of dATP, dCTP, dGTP, and dTTP.

As used herein, the phrase “standard conditions,” when used in reference to nucleic acid hybridization, refers to incubation at 55° C. in a buffer containing 15 mM Tris-HCl, pH 8.0, 50 mM KCl, and 2.5 mM MgCl₂, or its equivalent. Oligonucleotide primer molecules hybridized to a template nucleic acid (e.g., a Mycoplasma 16S rRNA gene or an internal amplification control template) under these conditions will be extended by at least one nucleotide by a template-dependent nucleic acid extending enzyme provided that the 3′-terminal two nucleotides of the primer are base paired to the template.

Unless otherwise specifically stated otherwise, as used herein, the terms “Mycoplasma,” “Mycoplasma species,” “Acholeplasma,” “Acholeplasma species,” and “Mycoplasma/Acholeplasma” are intended to include and encompass all members of the genus Mycoplasma and the genus Acholeplasma. The terms are used interchangeably, and use of one or another term is not intended to exclude the others, unless specifically stated.

As used herein, the phrase “cross-hybridizes” refers to the hybridization of an oligonucleotide primer designed to hybridize with a Mycoplasma species 1 6S rRNA gene sequence with a 16S rRNA from a non-Mycoplasma species.

As used herein, the phrase “does not base pair with” or “is mismatched” means that a given sequence of nucleotides on an oligonucleotide primer does not form complementary hydrogen bonds with an adjacent nucleotide sequence on a nucleic acid molecule. As the phrase is used herein, when one or more 3′-terminal nucleotides on an oligonucleotide primer “do not base pair” with a template nucleic acid molecule, a template-dependent nucleic acid extending enzyme will not extend the primer by one nucleotide or more under annealing and polymerization conditions as follows: 10 μCi of each of ³³P-labeled dATP, dCTP, dGTP, and dTTP (>1000 Ci/mMole), 1× Taq polymerase buffer (10 mM Tris-HCl, pH 8.8, 50 mM KCl, 1.5 mM MgCl₂, 0.001% (w/v) gelatin; or its equivalent), 100 nM of primer, 2.0 mM MgCl₂, 100 fmol template and 0.04 U/μl of Taq200™ polymerase (Stratagene #600197-51); the mixture is heated at 94° C. for 30 seconds, annealing is performed at 55° C. for 30 seconds, and polymerization is performed at 72° C. for one minute. The presence of one or more labeled species detected by autoradiography when the reaction products are separated on polyacrylamide gel demonstrates the extension of the primer. If there are no labeled species, the terminal nucleotide(s) of the primer “does not base pair with” the template. Alternatively, when the sequence of a potential contaminating template, e.g., an E. coli 16S rRNA gene sequence, is known, one can manually or via computer (e.g., using BLAST, with default parameters) align a given primer sequence with the contaminating template sequence. If one or more (e.g., one, two, three) of the 3′-terminal three nucleotides of the primer are not complementary to the template, they “do not base pair” with the template.

As used herein, the phrase “amplification control template” refers to a double- or single-stranded nucleic acid molecule that is added to a nucleic acid amplification reaction to serve as a control for the activity of the template-dependent nucleic acid extending enzyme used in such reaction. Various suitable control templates are known in the art. For example, an amplification control template useful according to the methods disclosed herein is mouse muscle nicotinic acetylcholine receptor, gamma-subunit, specific nucleic acid. This nucleic acid can be amplified using the primers disclosed herein. Amplification of the amplification control template can be distinguished from amplification of the target template by melting temperature or product length.

As used herein, the phrase “template-dependent nucleic acid extending enzyme” refers to an enzyme that catalyzes the template-dependent addition of nucleotides to the 3′ end of a nucleic acid strand hybridized to a substantially complementary template nucleic acid strand. A template-dependent nucleic acid extending enzyme useful in the methods disclosed herein will not extend an oligonucleotide primer in which one or more 3′-terminal nucleotides (e.g., the last 3′-terminal nucleotide, the last two 3′-terminal nucleotides, etc.) is not base paired with the template nucleic acid. That is, a template-dependent nucleic acid extending enzyme useful in the methods disclosed herein requires that at least the 3′ terminal two nucleotides of the primer strand be base paired with the template. Base pairing of the 3′-terminal two nucleotides of a primer with the template can be determined by alignment of the sequences, either manually or by computer. If the last one or two 3′ nucleotides of the primer are complementary to the template, the template-dependent nucleic acid extending enzyme useful in the methods described herein will extend the primer by at least one nucleotide, and preferably more, under conditions as described in the definition of “does not base pair,” above. If, on the other hand, the alignment shows that the last one or two nucleotides are not complementary to the template, a template-dependent nucleic acid extending enzyme useful in the methods described herein will not extend the primer by one or more nucleotides under the same conditions.

As used herein, the phrase “uracil DNA glycosylase enzyme” refers to a DNA repair enzyme that catalyzes the hydrolysis of uracil residues from single-stranded or double-stranded DNA. The removal of uracil residues from a DNA molecule leaves an abasic site rendering the DNA strand subject to cleavage by heat under alkaline conditions and non-functional as a template for amplification. Suitable uracil DNA glycosylase enzymes, e.g., E. coli uracil DNA glycosylase, are commercially available.

As used herein, the term “isolated” refers to a population of molecules, e.g., polypeptides, polynucleotides, or oligonucleotides, the composition of which is less than 50% (by weight), preferably less than 40% and most preferably 2%, 1%, 0.5%, 0.2%, 0.1%, or less, contaminating molecules of an unlike nature.

As used herein, the term “set” refers to a group of at least two. Thus, a “set” of oligonucleotide primers comprises at least two oligonucleotide primers.

As used herein, an “oligonucleotide primer” and a “primer” are used interchangeably in their most general sense to include any length of nucleotides which, when used for amplification purposes, can provide a free 3′ hydroxyl group for the initiation of DNA synthesis by a DNA polymerase, either using a RNA or a DNA template. DNA synthesis results in the extension of the primer to produce a primer extension product complementary to the nucleic acid strand to which the primer has hybridized. Generally, the primer comprises from 3 to 100 nucleotides, preferably from 5 to 50 nucleotides and even more preferably from 10 to 35 nucleotides. Primers are often selected to be any number of nucleotides between 10 and 25 nucleotides or more in length. The primers of the present invention may be synthetically produced by, for example, the stepwise addition of nucleotides or may be fragments, parts, portions or extension products of other nucleotide acid molecules. The term primer is used generally to encompass both strands of a given sequence (i.e., a given sequence and its complementary sequence).

“Complementary” refers to the broad concept of sequence complementarity between regions of two polynucleotide strands or between two nucleotides through base-pairing. It is known that an adenine nucleotide is capable of forming specific hydrogen bonds (“base pairing”) with thymine or uracil. Similarly, it is known that a cytosine nucleotide is capable of base pairing with a guanine nucleotide. This hydrogen bonding is the basis of the hybridization mentioned in this document.

As used herein, the phrase “extension product” refers to the nucleic acid product of an extension reaction catalyzed by a template-dependent nucleic acid extending enzyme. An “extension product” has been extended by at least one nucleotide by a template-dependent nucleic acid extending enzyme.

As used herein, the phrase “detectably different in size or sequence” means that the extension or amplification product formed by enzymatic extension or amplification of an internal amplification control template can be distinguished from the extension or amplification product of a target nucleic acid on the basis of a difference in size or sequence using techniques known to those of skill in the art or described herein. Conditions are well known for the separation of nucleic acids differing by as little as one nucleotide in length. Thus, the phrase “detectably different in size or sequence” means that a molecule differs by at least one nucleotide in length from another. It is preferred, however, that molecules of “detectably different” size differ by more than one nucleotide, e.g., by at least 10 nucleotides, 50 nucleotides, 100 nucleotides, or more. Alternatively, molecules of different sequence can be distinguished, e.g., on the basis of an enzymatic cleavage site or a binding site for a ligand that is present on one nucleic acid molecule but not on the other. Likewise, they can be distinguished based on their respective melting temperatures. This technique can be advantageous in the context of QPCR reactions because differences in melting temperatures can be determined without the need to prepare and run separate reactions, gels, etc. Thus, in the context of QPCR reactions, this technique can be easier to perform, generate less waste, and provide results faster than other techniques for detecting different sizes or sequences. Such molecules are thus of “detectably different” sequence.

As used herein, the term “silica gel” refers to such gels known in the art (e.g., as described in U.S. Pat. No. 4,923,978, the entirety of which is hereby incorporated by reference), which can be used to separate nucleic acids from other cellular components (e.g., proteins). One non-limiting example of a silica gel according to the invention includes the hydroxylated silica particles provided by Stratagene Inc. (La Jolla, Calif., Cat # 400714).

Unless otherwise defined in the present text, other terms and phrases are used in accordance with their art-recognized meanings.

### DETAILED DESCRIPTION OF CERTAIN EMBODIMENTS

Eight species of Mycoplasma, including Acholeplasma laidlawii, Mycoplasma arginini, M. fermentans, M. hominis, M. hyorhinis, M. orale, M. pirum, and M. salivarium (Tang, et al., 1999), account for greater than 95% of cell culture contamination. Acholeplasma laidlawii and M. pirum tend to be more difficult to detect than the other common species because they differ more widely from the other species. The assays described herein detect the presence of at least these eight species in a single assay. Further features of the assays disclosed herein include: 1) consistent amplification of genomic DNA (gDNA) from target Mycoplasma species, with as little as 10-100 copies of gDNA per PCR reaction; 2) the reagents and methods described permit, where desired, the production of a single PCR product of approximately the same size for all species of interest; 3) reduction or elimination of false positive assay results caused by the presence of E. coli nucleic acid in preparations of recombinant enzymes used to amplify target gene sequences; 4) prevention of carry-over contamination from previous assays; and 5) a robust, well characterized internal amplification control template to control for the presence of inhibitors of the amplification reaction.

In a first aspect, the invention provides nucleic acids that can be used in detecting Mycoplasma species, Acholeplasma species, or combinations of one or more species from these two genera. The nucleic acids can be oligonucleotides that can function as primers for PCR reactions. The nucleic acids can also be primers or genomic or sub-genomic nucleic acids that can be used as controls for monitoring the progression, specificity, and/or sensitivity of the methods of the invention.

Primers according to the invention can specifically hybridize to a Mycoplasma/Acholeplasma nucleic acids and permit template-dependent extension of the Mycoplasma/Acholeplasma nucleic acid during acellular amplification of the nucleic acid. Suitable primers can be designed by those of skill in the art based on known nucleic acid sequences of Mycoplasma/Acholeplasma species. Suitable primers can show perfect identity to the selected, known sequences, or can have less than 100% identity, as long as the identity is sufficient for the primer to specifically hybridize to the Mycoplasma/Acholeplasma target nucleic acid and permit extension of the primer under the appropriate conditions. Non-limiting examples of primers suitable for amplifying and detecting various Mycoplasma/Acholeplasma species are listed in Table 1.

TABLE 1

Exemplary Primers For Amplification/
Detection of Mycoplasma species

| | |
|---|---|
| Forward Primers (“F”) | Sequence: |

| M. spp | 5′-GACAGATGGTGCATGGTT-3′ | (SEQ ID NO:1) |
|---|---|---|
| M. arginini | 5′-GACAGATGGTGCATGGTT-3′ | (SEQ ID NO:1) |
| M. hominis | 5′-GACAGATGGTGCATGGTT-3′ | (SEQ ID NO:1) |
| M. fermentans | 5′-GACAGATGGTGCATGGTT-3′ | (SEQ ID NO:1) |
| M. orale | 5′-GACAGATGGTGCATGGTT-3′ | (SEQ ID NO:1) |
| M. salivarium | 5′-GACAGATGGTGCATGGTN-3′ | (SEQ ID NO:9) |
| M. pirum | 5′-GACAGGTGGTGCATGGTT-3′ | (SEQ ID NO:10) |
| M. hyorhinis | 5′-GACAGGTGGTGCATGGTT-3′ | (SEQ ID NO:10) |
| A. laidlawii | 5′-TACAGGTGGTGCACGGTT-3′ | (SEQ ID NO:3) |
| Reverse Primers (“R2”) | Sequence: | |
| M. spp | 5′-CATATTGCTTCTCTTTGTACCG-3′ | (SEQ ID NO:2) |
| M. arginini | 5′-CATATTGCTTCTCTTTGTACCG-3′ | (SEQ ID NO:2) |
| M. hominis | 5′-CATATTGCTTCTCTTTGTACCG-3′ | (SEQ ID NO:2) |
| M. salivarium | 5′-CATATTGCTTCTCTTTGTACCG-3′ | (SEQ ID NO:2) |
| M. orale | 5′-CATATTGCTTCTCTTTGTACCG-3′ | (SEQ ID NO:2) |
| M. hyorhinis | 5′-CATATTGCTTCTCTTTGTACCG-3′ | (SEQ ID NO:2) |
| M. pirum | 5′-GGATTCGCAACTGTTTGTAATG-3′ | (SEQ ID NO:5) |
| M. fermentans | 5′-CACTTCGCTTCTCTTTGTACCG-3′ | (SEQ ID NO:11) |
| A. laidlawii | 5′-CCTATCGCTTCTCTTTGTTCCA-3′ | (SEQ ID NO:4) |

PCR-based bacterial detection assays rely upon the ability of a set of primers specific for a given gene or nucleic acid sequence (or set of such sequences sharing common primer hybridization sequences) to direct the amplification of a target bacterial sequence from among a background of non-target sequences. Target bacterial genes are often selected to vary as widely as possible from other known sequences in order to ensure the specificity of the assay. However, the present invention contemplates methods (i.e., assays) that detect more than one species of a given genus. That is, the present invention contemplates detecting multiple members of the genus Mycoplasma or the genus Acholeplasma in the same assay. Therefore, the present invention targets a genomic sequence that is well conserved among the Mycoplasma/Acholeplasma.

For design of the present Mycoplasma/Acholeplasma primers, attention was focused on genes for which sequence data was known for a majority of the species of interest. The 16S rRNA gene was selected as a suitable target. The genomic 16S rRNA gene sequences are available from GenBank via the web site of the National Center for Biotechnology Information (via hypertext transfer protocol on the world wide web at ncbi.nlm.nih.gov/Genbank/) for eight of the most common Mycoplasma species that infect cell cultures: Acholeplasma laidlawii (NCBI ID#M23932), Mycoplasma arginini (NCBI ID#M24579), M. fermentans (NCBI ID#M24289), M. hominis (NCBI ID#M24473), M. hyorhinis (NCBI ID#M24658), M. orale (NCBI ID#M24659), M. pirum (NCBI ID#M23940), M. salivarium (NCBI ID#M24661), and E. coli (NCBI ID#2367315). Additional Mycoplasma 16S rRNA gene sequences are also available through GenBank. The highly conserved nature of the 16S rRNA gene sequences makes it possible to design small sets of primers (e.g., 2, 3, or 4 members) that recognize multiple (e.g., 2, 3, 4, 5, 6, 7, 8, or more) Mycoplasma/Acholeplasma species.

Although targeting this evolutionarily conserved sequence permits one to identify numerous species of Mycoplasma/Acholeplasma with a limited number of primers or primer sets, because stretches of sequences within this target might be conserved across other species, primers of the invention were designed such that they serve as primers for extension of Mycoplasma/Acholeplasma, but not for non-target species nucleic acids, which might be present in samples being tested or in recombinant enzyme preparations that are added to the primers to create an amplification mixture.

In essence, to minimize the occurrence of false positives (i.e., detection of non-target nucleic acids that might be present in reaction mixtures), the Mycoplasma/Acholeplasma primers of the present invention (e.g., SEQ ID NO:1-5) were designed so that they cannot serve as primers for extension of non-target sequences. Particular care was given to ensuring that at least the 3′-terminal nucleotide of the Mycoplasma/Acholeplasma primers were mismatched with the genomic sequence of the homologous E. coli 16S rRNA gene. By providing a mismatched base at the 3′-terminus of the Mycoplasma/Acholeplasma primers (as compared to non-target sequences), the likelihood of extension of non-target sequences (e.g., E. coli sequences) is significantly reduced or completely eliminated. Accordingly, the design of the Mycoplasma/Acholeplasma primers of the present invention avoids the amplification of any potential E. coli DNA contamination present in cloned DNA polymerase (e.g., Taq DNA polymerase), which is typically used for the amplification reaction.

To design primers that satisfy the above criteria, Mycoplasma/Acholeplasma 16S rRNA gene fragments that might serve as primer sequences were examined for the possible recognition of known prokaryotic (e.g., E. coli) and eukaryotic (e.g., mammalian) genome sequences. The alignment of the target 16S rRNA sequence from Mycoplasma/Acholeplasma with a homologous sequence from another organism can be performed by one of skill in the art manually. However, it is easier and often more accurate to align the sequences using any of a number of software programs that are widely available. For example, where a homologous sequence is already known, the “Blast 2 Sequences” program (bl2seq; Tatusova & Madden, 1999) can be used. The program is available through the NCBI website and can be used with default alignment parameters. This program produces the alignment of two given sequences using the BLAST engine for local alignment. Default parameters (for comparisons performed for this invention and for use with the BLASTN program only) are as follows: Reward for a match: 1; Penalty for a mismatch: −2; Strand option Both strands; open gap penalty 5; extension gap penalty 2; gap x_dropoff 50; expect 10.0; word size 11; and Filter (checked). In addition, BLAST analyses are performed against human and mouse genomes at a decreased stringency level (1000-fold increase over default expect value). Suitable sequences for use as Mycoplasma/Acholeplasma can be identified in this way.

In embodiments, the present invention provides a primer for amplification and detection of Mycoplasma/Acholeplasma species, which has a nucleotide sequence comprising the sequence of SEQ ID NO:1, SEQ ID NO:2, SEQ ID NO:3, SEQ ID NO:4, or SEQ ID NO:5. In embodiments, the primer has a nucleotide sequence that consists of the sequence of SEQ ID NO:1, SEQ ID NO:2, SEQ ID NO:3, SEQ ID NO:4, or SEQ ID NO:5.

In embodiments, the present invention provides a primer set, which comprises two or more primers for the amplification and detection of Mycoplasma/Acholeplasma species. The primer set can include combinations of two or more primers, each individually having a nucleotide sequence comprising the sequence of SEQ ID NO:1, SEQ ID NO:2, SEQ ID NO:3, SEQ ID NO:4, or SEQ ID NO:5. Thus, for example, a primer set according to the present invention can comprise two primers, one having a sequence comprising SEQ ID NO:1 and the other having a sequence comprising SEQ ID NO:2. Likewise, a primer set according to the present invention can comprise a primer having a sequence comprising SEQ ID NO:1 and the other having a sequence comprising SEQ ID NO:5. It is to be understood that primer sets are not limited to two primers. Rather, three or more can be present in a set. Exemplary Mycoplasma/Acholeplasma primers are presented in Table 1, above.

In a preferred embodiment, a primer set comprising a primer having a sequence comprising SEQ ID NO:1 and/or SEQ ID NO:3 and a primer having a sequence comprising SEQ ID NO:2, SEQ ID NO:5, and/or SEQ ID NO:4 is provided.

Primers according to the invention can also be specific for nucleic acids from organisms other than Mycoplasma/Acholeplasma. That is, primers according to the invention that are useful for detecting Mycoplasma/Acholeplasma species can be primers that are used to run control reactions, such as amplification controls (AC). As used herein, these are referred to as control primers. Non-limiting examples of control primers are listed in Table 2.

TABLE 2

EXEMPLARY PRIMERS FOR CONTROL REACTIONS

| | |
|---|---|
| Forward Primers: | Sequences: |

| β-actin | 5′-ATGGGTCAGAAGGATTCCTA-3′ | (SEQ ID NO:6) |
|---|---|---|
| E. coli 23S rRNA | 5′-GAAAGGCGCGCGATACAG-3′ | (SEQ ID NO:13) |
| Mouse muscle | 5′-TGGCAGAACTGTTCCCTCATCTTC-3′ | (SEQ ID NO:7) |
| nicotinic acetylcholine | | |
| receptor gamma- | | |
| subunit | | |
| Reverse Primers: | Sequences: | |
| β-actin | 5′-TCCATGTCGTCCCAGTT-3′ | (SEQ ID NO:8) |
| E. coli 23S rRNA | 5′-GTCCCGCCCTACTCATCGA-3′ | (SEQ ID NO:14) |
| Mouse muscle | 5′-GGGCGATGATGTTGATGACGTAGA-3′ | (SEQ ID NO:12) |
| nicotinic acetylcholine | | |
| receptor gamma- | | |
| subunit | | |

Although the primers can have a sequence that consists of the sequences presented herein, the primers can also contain other nucleotides, as long as the additional nucleotides do not destroy the ability of the primer to serve its function or reduce the specificity of the primer. While there is no absolute length requirement for primers of the invention, suitable primers typically are between 12 and 35 nucleotides in length. For example, suitable primers are often 15 or more nucleotides in length, 22 or more nucleotides in length, 25 or more nucleotides in length, or 30 or more nucleotides in length.

Primer syntheses can be carried out by any known method. For example, primers can be produced using cyanoethyl phosphoramidite chemistry, ammonium hydroxide deprotection, and desalting by gel filtration chromatography. If desired, primers can be further purified by high performance liquid chromatography (HPLC), polyacrylamide gel electrophoresis (PAGE), or any other method known to those of skill in the art.

Nucleic acids other than primers are also part of this invention. These nucleic acids can be used as controls for monitoring various aspects of the methods of the present invention. The control nucleic acids can be Mycoplasma/Acholeplasma nucleic acids, which can be used as positive controls to confirm that the methods and primers are suitable for amplification and detection of Mycoplasma/Acholeplasma nucleic acids. The control nucleic acids can also be other bacterial nucleic acids or eukaryotic nucleic acids. These other nucleic acids can be used to confirm the specificity of the primers used to amplify and detect Mycoplasma/Acholeplasma nucleic acids and to confirm that no inhibitors of amplification were present in the amplification mixtures. Control nucleic acids can be genomic or sub-genomic nucleic acids.

In embodiments, genomic or sub-genomic nucleic acids from M. orale and/or A. laidlawii are provided. These nucleic acids can be used in the methods of the invention as positive control templates to validate that polymerase-mediated amplification of Mycoplasma/Acholeplasma templates can be successfully detected. In other embodiments, genomic or sub-genomic nucleic acids from one or more other Mycoplasma and/or Acholeplasma are provided as positive control templates.

In embodiments, eukaryotic sub-genomic nucleic acids are provided to enable the practitioner to confirm that the methods of the invention were not inhibited by some substance, such as one present in the sample being tested. As used herein, these nucleic acids, and the amplification reactions that participate in, are called amplification controls (AC). A non-limiting example of a suitable genomic or sub-genomic nucleic acid is nucleic acid encoding the mouse muscle nicotinic acetylcholine receptor gamma-subunit, or a portion thereof, the sequence for which is publicly available.

In other embodiments, prokaryotic nucleic acids are provided. In these embodiments, the prokaryotic nucleic acids contain sequences that are homologous to the target Mycoplasma/Acholeplasma sequences. These prokaryotic nucleic acids can be used to assess the specificity of the Mycoplasma/Acholeplasma primers. Exemplary nucleic acids for these embodiments are nucleic acids encoding all or part of the E. coli 16S rRNA gene.

Although the specificity of the primers can be determined using prokaryotic nucleic acids, it can also be assessed without the addition of these nucleic acids because recombinantly produced polymerases suitable for use in the present methods typically contain contaminating prokaryotic nucleic acids, which can serve as the control nucleic acid. Thus, even in the absence of addition of, for example, E. coli DNA, the specificity of the Mycoplasma/Acholeplasma primers can be determined simply by adding a recombinantly produced polymerase suitable for PCR, such as Taq polymerase.

Use of control nucleic acids, and exemplary nucleic acids are discussed in more detail below.

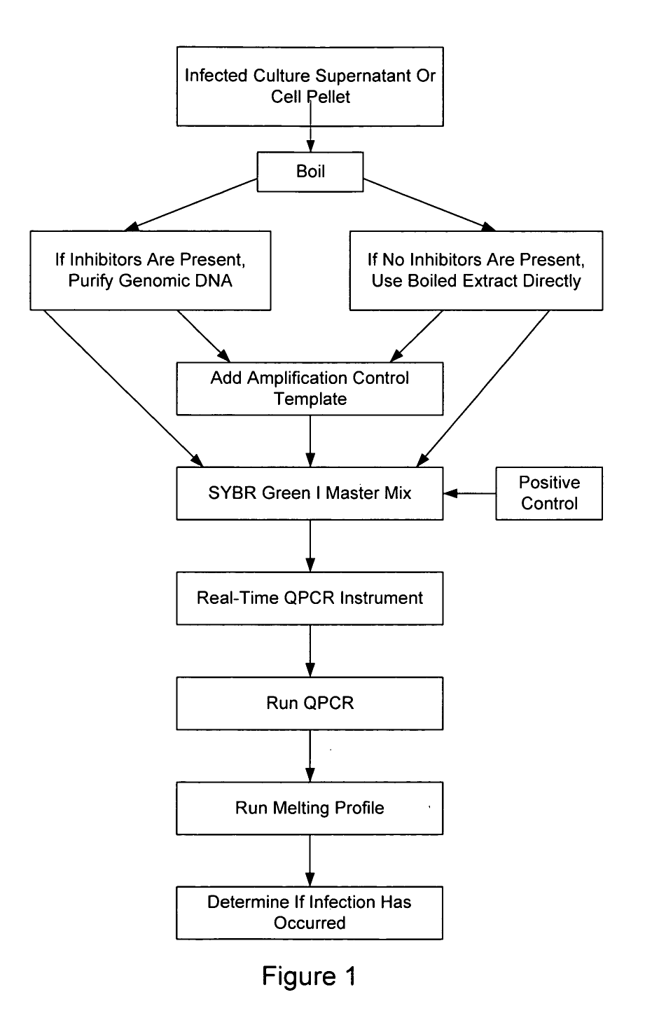

In a second aspect, the invention provides methods of acellular amplification of target nucleic acids. The methods of the invention use at least two oligonucleotides to specifically amplify Mycoplasma and/or Acholeplasma nucleic acids, while essentially avoiding amplification of nucleic acids from other bacteria or eukaryotes (except as specifically intended when those nucleic acids are used for control reactions). In general, the methods comprise providing a purified target nucleic acid, and amplifying and detecting target sequences within the target nucleic acid. FIG. 1 shows exemplary embodiments of the methods of the invention, which will be discussed below.

Purified target nucleic acids can be provided in any state of purification. However, it is preferred that the nucleic acids be provided in as purified state as possible. As with most, if not all, acellular amplification reactions, it has been found that the sensitivity and reproducibility of the methods of the present invention are improved, generally, as the purity of the target nucleic acid is increased. For example, reduction or elimination of substances present in culture media, such as those containing fetal calf serum, metabolic products, cell debris, or antibiotics, has been found to improve the results of the present methods.

Thus, in embodiments, the methods include purifying the target nucleic acid prior to amplification. Numerous suitable purification protocols for nucleic acids are known in the art. For example, the StrataPrep® PCR Purification Kit (Catalog # 400771, Stratagene, La Jolla, Calif.) can be used to purify the target nucleic acid prior to amplification. Alternatively, a silica gel (e.g., StrataClean® Resin, Cat. No. 400714, Stratagene, La Jolla, Calif.) may be used to reduce the concentration of components in the sample that are inhibitory to PCR amplification. The practitioner may select any suitable protocol or modify a known protocol to optimize the amplification of the target nucleic acid sequences, based on the source of the nucleic acid, the amount of nucleic acid present in the source, or any other variable that might affect purification quality and quantity. Such modifications are well within the skill of the skilled artisan, and can be implemented without undue or excessive experimentation.

The methods of the invention include amplifying target nucleic acids to permit detection of Mycoplasma infection of a sample. Although any acellular amplification method may be suitable, quantitative PCR (QPCR) (also referred as real-time PCR) is preferred under some circumstances because it provides not only a quantitative measurement, but also reduces the amount of time required to obtain a result, and reduces contamination. Two types of QPCR detection are common: unspecific detection (for example SYBR® Green I) and probe-based detection (for example TaqMan, Molecular Beacons, Scorpions, etc.). In order to develop a real-time Mycoplasma detection kit, gel read-out is used in embodiments to aid in determining if the PCR is optimized.

It is further contemplated that other enzyme-mediated amplification assays, for example 3SR (Self-Sustained Sequence Replication; Gingeras et al., Annales de Biologie Clinique, 48(7):498-501, 1990; Guatelli et al., Proc. Natl. Acad. Sci. U.S.A., 87:1874, 1990), or SDA (Strand Displacement Amplification; Walker, Nucleic Acids Res. 22:2670-2677, 1994), can also be used in the present methods. The presence of contaminating recombinant host nucleic acid will pose the same false-positive problems in any such system that is dependent upon the extension of a hybridized primer for its signal and for its specificity. Thus, the present methods will be equally advantageous for these assay formats as for QPCR.

Suitable conditions for acellular amplification reactions are known in the art and can be applied or modified by those of skill in the art to optimize reactions to achieve desired goals. Thus, variations on the exact amounts of the various reagents and on the conditions for the PCR (e.g., buffer conditions, cycling times, etc.) that lead to similar amplification or detection results are known to those of skill in the art or readily identified, and are considered to be equivalents.

Thus, in embodiments, the present invention provides a method of acellular amplification of Mycoplasma or Acholeplasma nucleic acids. The method comprises providing a sample suspected of containing a purified nucleic acid from Mycoplasma or Acholeplasma; providing at least two oligonucleotide primers, each of these primers having a sequence comprising the sequence of SEQ ID NO:1, SEQ ID NO:2, SEQ ID NO:3, SEQ ID NO:4, or SEQ ID NO:5; amplifying the purified Mycoplasma or Acholeplasma nucleic acid; and detecting the product of the amplifying reaction. Embodiments of the methods specifically detect Acholeplasma laidlawii, Mycoplasma arginini, M. fermentans, M. hominis, M. hyorhinis, M. orale, M. salivarium, M. pirum, or a combination of two or more of these organisms.

As discussed above, in embodiments, the present method includes a quantitative PCR (or a real time QPCR) method for Mycoplasma detection. As used herein, “quantitative PCR (or a real time QPCR)” refers to the direct monitoring of the progress of a PCR amplification as it is occurring, without the need for repeated sampling of the reaction products. In quantitative PCR, the reaction products may be monitored as they are generated and are tracked after they rise above background but before the reaction reaches a plateau. The number of cycles required to achieve a chosen level of fluorescence varies directly with the concentration of amplifiable targets at the beginning of the PCR process, enabling a measure of signal intensity to provide a measure of the amount of target DNA in a sample in real time.

Quantitative PCR according to the present invention may be performed on any suitable instrument, including, but not limited to, Mx4000 or Mx3000P (Stratagene, La Jolla, Calif.), ABI7700 or ABI7000 (Applied BioSystems Inc., Foster City, Calif.), MJ Opticon (MJ Research), iCycler (Biorad), RotorGene 3000 (Corbett), and the SmartCycler (Cepheid).

The present invention encompasses QPCR detection methods having a sensitivity of detecting less than 500 copies (preferably less than 250 copies, more preferably less than 100 copies, still more preferably less than 50 copies) of genomic Mycoplasma DNA in a sample. Indeed, under optimal conditions, as few as 10 copies or even one copy of a Mycoplasma or Acholeplasma genome can be detected.

In embodiments, a hot-start PCR reaction is performed. In such situations, a hot start Taq DNA polymerase, such as SureStart® Taq DNA polymerase from Stratagene, can be used to improve PCR reaction by decreasing background from non-specific amplification and to increase amplification of the desired extension product.

As mentioned above, in embodiments, at least a portion of the amplification products is analyzed using gel electrophoresis. Gel electrophoresis can be used to evaluate the amplification reaction prior to amplification using QPCR. Gel readout can be used to optimize QPCR reaction parameters by permitting one to identify successful PCR conditions, and permitting one to confirm that the amplified product is the correct size. That is, the presence of an amplified band of an expected size detected after gel electrophoresis of PCR amplification products confirms the presence of the target bacterium or genus of target bacteria. It also permits the user to qualitatively, semi-quantitatively, or quantitatively assess the amplification reaction. In these embodiments, the results of the gel can aid the user in making suitable minor changes to the amplification reaction conditions or identify potential problems, such as insufficiently pure starting materials. Upon confirming by gel readout that amplification has successfully occurred, the user can continue with the general method of the invention by detecting amplified product, or can repeat the amplification process using the parameters that were identified as suitable for amplification. In these embodiments, gel readout is used to optimize the real-time Mycoplasma detection assay.

Thus, the present invention provides a method of QPCR amplification and detection of target Mycoplasma/Acholeplasma sequences to detect infection of a sample with one or more Mycoplasma or Acholeplasma bacteria, where the QPCR amplification/detection uses SYBR® (Molecular Probes, Eugene, Oreg.) Green dye, the Brilliant® SYBR® Green QPCR Master Mix (Stratagene, La Jolla, Calif.), and, optionally, the ROX passive reference dye. The method comprises purifying nucleic acids from a sample, and performing QPCR with a Mycoplasma primer set of the invention. In embodiments, the method further comprises amplifying Mycoplasma/Acholeplasma control gDNA with the same primer set used to test for Mycoplasma/Acholeplasma in the sample being tested. In exemplary embodiments, in a separate reaction, a known amount of purified M. orale control gDNA is amplified. In exemplary embodiments, in a separate reaction, a known amount purified A. laidlawiigDNA is amplified. In yet other embodiments, the method comprises, in addition to performing QPCR with a Mycoplasma primer set of the invention, amplifying Mycoplasma/Acholeplasma control gDNA, or both, amplifying an amplification control (AC) template with two AC primers. In embodiments, the AC template is simultaneously amplified with the Mycoplasma in the sample in the same reaction tube. In other embodiments, the Mycoplasma sample is amplified in a separate reaction tube than the AC, but under the same reaction conditions. In embodiments, the AC is mouse muscle nicotinic acetylcholine receptor, gamma subunit. In other embodiments, the AC is a human β-actin sequence. In certain embodiments, all nucleic acids that are amplified are purified before being subjected to amplification. In yet further embodiments, amplification is analyzed and, optionally, quantitated by generating a standard curve and comparing the sample results to the standard curve.

The primary real-time method of detecting amplified product in a QPCR reaction according to the present invention is by detecting fluorescence associated with the production of dsDNA. As dsDNA accumulates, the amount or intensity of fluorescence increases. The amount of fluorescence is detected by the QPCR instrument, and the data can be plotted to create an amplification plot. The data can also be used to derive the threshold cycle (Ct), from which the initial copy number of the target may be quantified, e.g., as described in Higuchi et al., Biotechnology (N Y) 11(9):1026-30, 1993. Here, Ct is defined as the cycle at which fluorescence is statistically significant above background, i.e., the noise of the background is calculated first (equals 1 sigma), after which 10×sigma is calculated and a corresponding line is drawn (the threshold line). The Ct is defined as the point at which the fluorescence crosses the threshold line. The threshold cycle is inversely proportional to the log of the initial copy number (e.g., Higuchi et al.). The more template that is initially present, the fewer the number of cycles required for the fluorescence signal to become detectable above background.

The methods of the present invention can also include monitoring or detecting amplification of target and/or control sequences using the different melting temperatures of amplification products. That is, in addition to detecting fluorescence due to dsDNA by way of an amplification plot, the presence and identify of PCR products can be monitored by assaying the dissociation of dsDNA in the reaction mixture. In general, when detecting dsDNA amplification products, samples are first denatured at high temperature, then allowed to re-anneal. They are then subjected to a stepwise increase in temperature from about 55° C. to about 95° C., with fluorescence measurement taken periodically, for example at each temperature increment. As the temperature increases, the amplification products in each tube will melt according to their composition. If primer-dimer or nonspecific products were made during the amplification step, they will generally melt at a lower temperature (Tm) than the desired products. The melting of products results in SYBR® Green dissociation from the nucleic acids, which results in decreased fluorescence. After data collection is complete, fluorescence is plotted versus temperature. For an easy interpretation of the dissociation profile the first derivative of fluorescence should be displayed, i.e. —R′(T) or —Rn′(T).

Dissociation curves are typically generated by following the instrument manufacturer's guidelines for setting up a dissociation curve. For example, on an Mx3000P instrument (Stratagene), the positive control reaction typically produces an early Ct (˜26) during amplification and a dissociation curve peak with Tm about 82° C., indicating that the experiment is working properly. The negative control reaction produces no or little Ct during amplification and no or little peaks in the dissociation curve. If a culture is contaminated with Mycoplasma (>10-50 cell equivalents per sample), a Ct value (<30) will typically be observed in the amplification data and the dissociation curve will include a peak at Tm around 82° C. For cell culture reactions spiked with the Amplification Control template exemplified in the present text, a Ct value (<30) should be observed in the amplification data and the dissociation curve should include a Tm peak around 85° C. Failure to obtain a Ct value and a dissociation Tm peak around 85° C. indicates that the sample may contain agents inhibitory to the PCR amplification.

In conjunction with a dissociation curve, a standard curve can be generated from QPCR data. The standard curve can provide information about the efficiency of amplification of the target nucleic acid, the concentration range through which linear amplification occurs in the method, and, not least importantly, the quantity of nucleic acids detected in a sample. For example, a series (e.g., serial dilutions) of known quantities of purified gDNA from an organism can be run, and QPCR amplification data collected. This data can then be used to generate a standard curve for detection of that DNA. A sample containing an unknown number of organisms can then be analyzed, and the number of copies of DNA detected can be determined using the standard curve. This information, coupled with information about the efficiency of isolation of DNA from the sample, can permit the user to determine the total number of organisms present in the original sample.

The methods of the invention can include providing and amplifying nucleic acids other than those of the target Mycoplasma or Acholeplasma. Such other nucleic acids act as controls for the methods, and can be purified by any known technique, for example by use of any of the kits discussed above. These other nucleic acids can be used as controls to assess the specificity of the PCR reaction for Mycoplasma/Acholeplasma, and to detect the presence of PCR inhibitors in the sample being tested. Suitable other nucleic acids include other prokaryote nucleic acids or eukaryote nucleic acids. In embodiments, nucleic acids encoding at least a portion of the mouse muscle nicotinic acetylcholine receptor gamma-subunit, and Mycoplasma and Acholeplasma nucleic acids are used as controls.

Controls according to the invention are of three types: a negative control, a positive control, and an amplification control (AC). Broadly, controls are methods of amplifying nucleic acids to obtain information about the performance of amplification and detection of target nucleic acids. The controls include primers for amplification reactions, and methods of amplifying nucleic acids. The controls can also include template nucleic acids that are specifically amplified by the primers. Although the methods of the invention can be performed without performing any of the controls, performing one, two, or all three of the controls can provide the practitioner with information that can be advantageous under some circumstances, such as when the method detects the presence of target nucleic acids when the practitioner did not expect such a result, when the method fails to provide the expected results, or when the method provides the expected results, but fails to provide the quality or quantity of results expected.

The first control is a negative control to confirm that the primers used for amplification and detection of Mycoplasma/Acholeplasma do not amplify non-target sequences that might be present in the amplification/detection reaction mixture. The negative control reaction contains all of the reagents, primers, solutions, etc. that are present in the test reaction except the sample to be tested. Although not required by the present methods, to ensure the most valid results, the negative control reaction should be run the same way that the test reaction is run, using the same thermocycler and same amplification program. Results of the negative control reaction can be monitored in any known way. In embodiments, the results are monitored by increased fluorescence in a QPCR reaction. In embodiments, the results are monitored by analysis of gel electrophoresis of at least a portion of the amplification reaction mixture after amplification has been performed. In the latter method, a band on the gel indicates the presence of contaminating nucleic acids in one or more of the reagents, solutions, etc. used for the amplification reaction.

The second control is a positive control. The positive control uses a 16S rRNA gene, or portion thereof, from a Mycoplasma or Acholeplasma species, and an amplification protocol. This control contains sequences to which the Mycoplasma/Acholeplasma primers should hybridize, and prime amplification of the control nucleic acid. This control is used to confirm that the Mycoplasma/Acholeplasma primers hybridize with the target sequences and prime amplification of the target sequences, and to confirm that amplification of target Mycoplasma/Acholeplasma nucleic acid can be detected by the methods of the invention. As with the other controls, although not required by the present methods, to ensure the most valid results, the positive control reaction should be run the same way that the test reaction is run, using the same thermocycler and same amplification program.

The third control is an amplification control (AC). The amplification control uses a nucleic acid from a non-target species, and at least two control primers, along with an amplification protocol. The control primers are designed to act as specific primers for amplification of the AC nucleic acid under the amplification protocol used, and to produce a double stranded product of known size, which can be detected using standard techniques. One advantage of this positive control is to reduce the occurrence of false negative results in the methods of the invention. More specifically, when the AC is run under the same conditions as the test sample, production of an amplification product of the expected size and quantity indicates that the amplification method is suitable for amplifying nucleic acids. Thus, a lack of an amplification product in the test sample indicates the lack of target nucleic acids in the sample. However, the lack of an amplification product in the AC reaction indicates that at least one inhibitor of amplification is present in the reaction mixtures, and the potential for the results for the sample being a false negative.

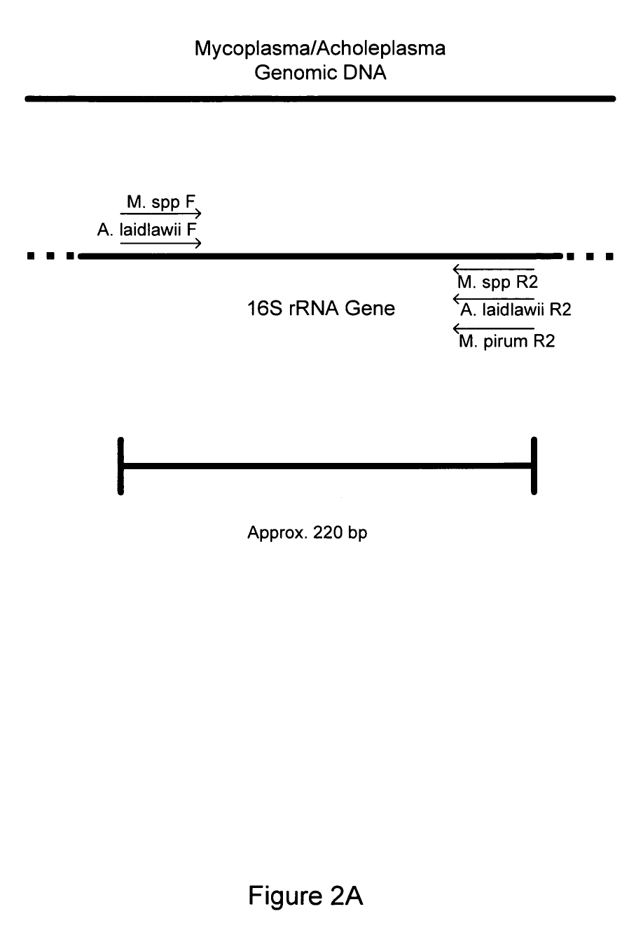

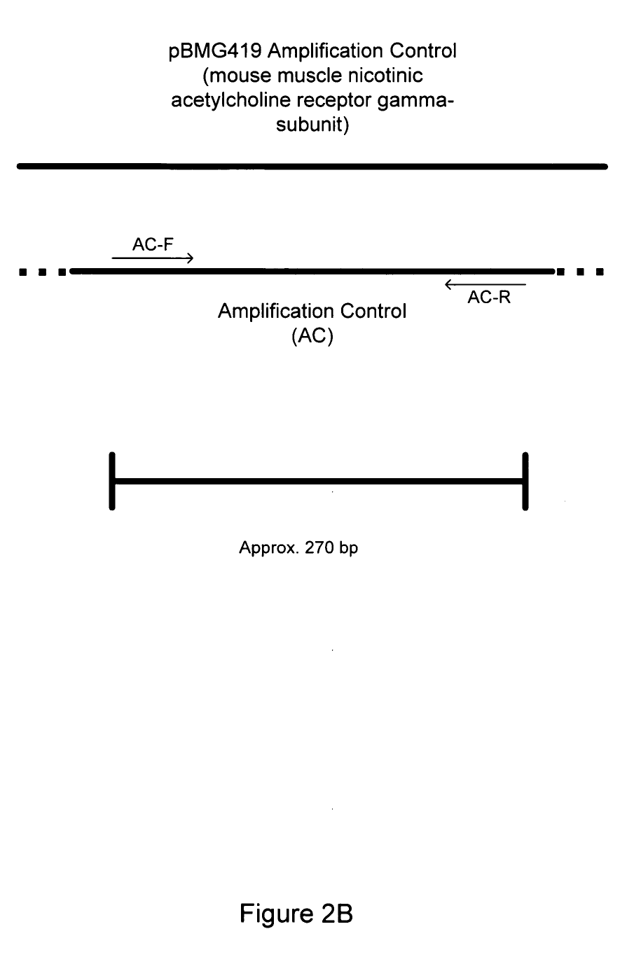

Various non-limiting exemplary primers and control templates are presented in FIG. 2.

The methods of the invention contemplate running the AC as a separate reaction in a separate tube from the sample reaction (“two tube format”). In this embodiment, two reactions are set up to run in parallel, in two separate reaction vessels. The methods of the invention also contemplate running the AC as a reaction in the same tube as the sample reaction (“single tube format”). In either embodiment, the AC, if amplified, will produce a double stranded nucleic acid of a known size (based on the placement of the control primers), which can be detected by any known technique, including gel electrophoresis and melting temperature, which is described in more detail below.

Accordingly, some embodiments of the invention include providing an amplification control nucleic acid and at least two primers that specifically hybridize to the amplification control; amplifying the amplification control; and detecting the product of the amplification control amplifying reaction. In certain embodiments, the amplifying and detecting of the purified Mycoplasma or Acholeplasma nucleic acid, and the amplifying and detecting of the amplification control are performed in a different reaction tube, while in other embodiments, the amplifying and detecting of the purified Mycoplasma or Acholeplasma nucleic acid and the amplifying and detecting of the amplification control are performed in the same reaction tube.

As discussed above, the methods can include monitoring or detecting amplification of target sequences and/or control sequence in real-time (i.e., QPCR) using a dye that detects double stranded nucleic acids to a much greater extent than single stranded nucleic acids or free nucleotides. The methods can also include monitoring or detecting amplification of target sequences and/or control sequences using other techniques, such as gel electrophoresis. Thus, the detection of the extension product in an acellular amplification (e.g., PCR or QPCR) may be performed by any methods described herein or known in the art, such as by polynucleotide staining or through a detectable label by using a labeled primer for the amplification.

In some embodiments, a polynucleotide stain is used due to its preferential staining for double stranded DNA. Thus the amount of extension products is reflected by the amount of stain signal produced. The use of such stains greatly decreases the cost and complexity of the reactions. The present invention can work well with such stains, since the objective is to measure the incorporation level of the stain relative to a standard. The polynucleotide stain is selected to have the desired relative polynucleotide binding affinity and spectral characteristics, according to methods well known in the art. While fluorescent stains are preferred stain for the present invention, any polynucleotide stain that emits light (including chemiluminescence or phosphorescence) is also useful. Dyes are useful not only for detecting amplification during QPCR reactions, but for embodiments of the invention where gel read-out is used to identify and evaluate reaction products during optimization of the amplification reaction.

Useful polynucleotide stain may be a phenanthridinium dye, including monomers or homo- or heterodimers thereof, that give an enhanced fluorescence when complexed with polynucleotides. Examples of phenanthridinium dyes include ethidium homodimer, ethidium bromide, propidium iodide, and other alkyl-substituted phenanthridinium dyes.

Useful polynucleotide stains may be or may incorporate an acridine dye, or a homo- or heterodimer thereof, such as acridine orange, acridine homodimer, ethidium-acridine heterodimer, or 9-amino-6-chloro-2-methoxyacridine.

Useful polynucleotide stains may also be an indole or imidazole dye, such as Hoechst 33258, Hoechst 33342, Hoechst 34580 (Molecular Probes, Inc. Eugene, Oreg.), DAPI (4′,6-diamidino-2-phenylindole), or DIPI (4′,6-(diimidazolin-2-yl)-2-phenylindole).

Useful polynucleotide stains may also be a cyanine dye or a homo- or heterodimer of a cyanine dye that gives an enhanced fluorescence when associated with polynucleotides. Any of the dyes described in U.S. Pat. No. 4,883,867 to Lee, U.S. Pat. No. 5,582,977 to Yue et al., U.S. Pat. No. 5,321,130 to Yue et al., and U.S. Pat. No. 5,410,030 to Yue et al. may be used, including polynucleotide stains commercially available under the trademarks TOTO, BOBO, POPO, YOYO, TO-PRO, BO-PRO, PO-PRO and YO-PRO from Molecular Probes, Inc., Eugene, Oreg. Likewise, any of the dyes described in U.S. Pat. No. 5,436,134 to Haugland et al., U.S. Pat. No. 5,658,751 to Yue et al., and U.S. Pat. No. 5,863,753 to Haugland et al. may be used, including polynucleotide stains commercially available under the trademarks SYBR, SYTO, SYTOX, PICOGREEN, OLIGREEN, and RIBOGREEN from Molecular Probes, Inc. (Eugene, Oreg.).

Useful polynucleotide stains may also be a monomeric, homodimeric or heterodimeric cyanine dye that incorporates an aza- or polyazabenzazolium heterocycle, such as an azabenzoxazole, azabenzimidazole, or azabenzothiazole, that gives an enhanced fluorescence when associated with polynucleotides. This includes, but is not limited to, polynucleotide stains commercially available under the trademarks SYTO, SYTOX, JOJO, JO-PRO, LOLO, LO-PRO from Molecular Probes, Inc., (Eugene, Oreg.).

Other useful polynucleotide stains include, but are not limited to, 7-aminoactinomycin D, hydroxystilbamidine, LDS 751, selected psoralens (furocoumarins), styryl dyes, metal complexes such as ruthenium complexes, and transition metal complexes (incorporating Tb3+ and Eu3+, for example).

A preferred stain used in some embodiments of the invention is SYBR® Green I, which is commercially available from Molecular Probes Inc., Eugene, Oreg. This stain is suitable for use in QPCR assays.

In a preferred embodiment, a passive reference dye is optionally used to normalize for non-amplification related fluorescence signal variation. A passive reference dye does not take part in the amplification reaction and its fluorescence remains constant during the reaction. The passive reference dye, therefore, provides an internal reference to which the amplification related signal can be normalized during data analysis. This is useful to correct for fluorescent fluctuations due to changes in concentration or volume in the wells. Normalization using passive reference dye is known in the art and it can be accomplished using appropriate analysis software, which divides the emission intensity of the reporter dye (e.g., a polynucleotide stain) by the emission intensity of the passive reference to obtain a ratio defined as the Rn (normalized reporter) for a given reaction well. The difference between the Rn value of a reaction containing all components including the template (Rn+), and the Rn value of an unreacted sample (i.e., no production of extension product, Rn−) equals the ΔRn value, which reliably indicates the magnitude of the signal generated by the given set of PCR conditions.

In one embodiment, ROX passive reference dye is used in addition to a polynucleotide stain (e.g., SYBR® Green). The excitation and emission wavelengths of the reference dye are 584 nm and 612 nm, respectively. The ROX dye can be provided as a concentrated solution dissolved in a buffer that is compatible with the PCR reaction buffer. The amount of the ROX passive reference dye can be adjusted based on the particular requirements of different instruments. In a preferred embodiment, SYBR® Green I is used to stain the extension product and ROX passive reference dye is used to normalize the signal generated by SYBR® Green. It is recommended that the use of ROX passive reference dye follows guidelines for passive reference dye optimization for each instrument used. For Stratagene's Mx3000P or Mx4000 instruments, ROX passive reference dye can be used at a final concentration of 30 nM according to one embodiment. For ABI real-time fluorescence detection platforms, such as the PRISM 7700 or the GeneAmp 5700, ROX passive reference dye can be used at a final concentration of 300 nM according to another embodiment. In one embodiment, for instruments that allow excitation at ˜584 nm (including most tungsten/halogen lamp-based instruments and instruments equipped with a ˜584 nm LED), optimization can begin by using the reference dye at a final concentration of 30 nM. In another embodiment, for instruments that do not allow excitation near 584 nm, (including most laser-based instruments), optimization can begin by using the reference dye at a final concentration of 300 nM.

SYBR® Green I dye has a high binding affinity to the minor groove of double-stranded DNA (dsDNA). It has an excitation maximum at 497 nm and an emission maximum at 520 nm. In the unbound state the dye exhibits little fluorescence; however, when bound to dsDNA, the fluorescence greatly increases, making it useful for the detection of product accumulation during real-time PCR. More specifically, during the denaturation step of PCR, all DNA becomes single-stranded. At this stage, SYBR® Green is free in solution and produces little fluorescence. During the annealing step, the primers will hybridize to the target sequence, resulting in dsDNA to which SYBR® Green I can bind. As the PCR primers are extended in the elongation phase, more DNA becomes double-stranded (e.g., as extension products), and a maximum amount of SYBR® Green I is bound. The increase in fluorescence signal intensity depends on the initial concentration of target present in the PCR reaction.

One consideration when using SYBR® Green I, however, is that signal can also be generated from nonspecific dsDNA (e.g., primer-dimers (PD) and spurious PCR products). The fluorescence resulting from amplification of the target will not be initially distinguishable from fluorescence attributable to the spurious PCR products. To distinguish between fluorescence derived from specific and non-specific products, the present method contemplates embodiments comprising a dissociation curve. During the dissociation curve, dsDNA is melted into ssDNA, for example by a stepwise increase in temperature or a linear increase in temperature, with fluorescence data collected at each step or continuously through the linear increase in temperature. The dissociation curve fluorescence data is analyzed to reveal the temperature(s) at which major populations of dsDNA are converted to ssDNA (i.e., the major Tm peaks). For example, the Mycoplasma amplicons amplified using the primers according to one embodiment of the present invention have a Tm of ˜82° C. In contrast, fluorescence due to PD displays a Tm of <75° C. (e.g., 74° C.), and spurious PCR products typically show a broad Tm.

Note that a common problem in SYBR® Green QPCR is the formation of primer dimers. Primer dimers derive from primers in a reaction that anneal and can be extended in PCR. The extended PD bind SYBR® Green and produce a signal. The dissociation curve of the present invention aids the practitioner in evaluating if PD are present in the reaction. PD typically melt at a lower temperature than the bona fide product, and thus can be distinguished from the product. Typical dissociation curves of the present invention (when target Mycoplasma nucleic acids are present in a sample) will contain a major peak for the Mycoplasma target at about 82° C. They will also contain a major peak at a higher temperature (Tm of about 85° C.), corresponding to the amplification control (AC). If a third peak is present, it will typically be a minor peak at about 74° C., representing PD. The absence of a Tm peak around 74° C. generally indicates the absence of primer dimers.

One problem encountered when laboratories routinely use the same primer sets for assays, such as the Mycoplasma detection assays described herein, is that small amounts of the amplified products from previous assays can contaminate subsequent reactions, giving false positive results. To avoid this problem, the Mycoplasma detection assays described herein can be routinely carried out in the presence of dUTP, which permits the user to eliminate carry-over PCR products with uracil DNA-glycosylase (UDG). In the event that amplification product is inadvertently carried over from one experiment to another, the enzyme UDG will catalyze the release of free uracil found in the contaminating product and hydrolysis of the DNA strand. A pre-incubation of the PCR reaction for 10 minutes at 37° C. activates the UDG enzyme. Heat energy in a subsequent denaturation step (e.g., 10 minutes at 94° C.) eliminates the UDG activity, activates the DNA polymerase (if modified for “hot-start” activation, e.g., SureStart® Taq), and catalyzes the cleavage of the contaminating abasic phosphodiester backbone. UDG is commercially available, e.g., from New England Biolabs (Cat. # M0280S).

The methods of the invention provide high specificity of PCR-based bacterial assays. More specifically, the methods have a very low frequency of false positive results. A common source of false positive results in PCR-based bacterial assays that use recombinant polymerase is the recombinant polymerase, which is often contaminated with genomic DNA from the host bacterium (typically E. coli). When the host bacterium has a homologous sequence to the target gene sequence in the bacterial genus or species being detected, the use of recombinant preparations of recombinant polymerase, for example Taq polymerase, often results in false positive amplification results when the primers cross-hybridize and permit extension from contaminating host species genomic DNA template.

The high specificity (i.e., lower false positive rates) of the present methods is achieved, at least in part, by the selection of PCR primer sequences that will not result in extension of the primer even if the primer, as a whole, can hybridize with contaminating template nucleic acid from the recombinant polymerase host species or other contaminating nucleic acids. In general, primer sequences are selected to have at least the 3′ terminal nucleotide of the primer mismatched with the E. coli genomic sequence that is homologous with the Mycoplasma or Acholeplasma target sequence. In this way, extension of the primer from any contaminating E. coli nucleic acids is minimized or eliminated, thus reducing or eliminating non-specific amplification of non-target nucleic acids (and thus false positive results).

It is also noted that the present methods can be used whenever a recombinant enzyme produced in bacteria, including a non-polymerase recombinant enzyme, is used in a mixture that is ultimately subjected to a PCR amplification of a target gene sequence from a different bacterial species. Thus, if, for example, a recombinant uracil DNA glycosylase or other recombinant enzyme is used in treatment or pre-treatment of a sample to be subjected to amplification, this approach will avoid false positive signal from recombinant host nucleic acid introduced with that recombinant enzyme.

In a third aspect, the invention provides compositions. In general, the compositions comprise at least two oligonucleotide primers that can be used to specifically amplify Mycoplasma and/or Acholeplasma nucleic acids. The compositions can also contain some or all of the reagents, solvents, and other nucleic acids for practicing the methods of the invention. The compositions can also include primers for performing control reactions. Likewise, the compositions can comprise genomic or sub-genomic nucleic acids that are suitable for use as control templates. Thus, in embodiments, compositions of the invention comprise at least one Mycoplasma specific primer and at least one Mycoplasma gDNA. Other embodiments include nucleic acids for the AC aspect of the invention. Accordingly, the compositions of the invention can contain a primer set for amplification of Mycoplasma/Acholeplasma target sequences, positive control Mycoplasma/Acholeplasma nucleic acids, a primer set for amplification of mouse muscle nicotinic acetylcholine receptor gamma-subunit, and mouse muscle nicotinic acetylcholine receptor gamma-subunit nucleic acid.

Thus, in embodiments, the composition comprises at least one oligonucleotide primer, each of these primers having a sequence comprising the sequence of SEQ ID NO:1, SEQ ID NO:2, SEQ ID NO:3, SEQ ID NO:4, or SEQ ID NO:5. For example, the composition can comprise a primer having a sequence comprising SEQ ID NO:1 and a primer having a sequence comprising SEQ ID NO:2. The composition can further comprise a primer having a sequence comprising SEQ ID NO:3, a primer having a sequence comprising SEQ ID NO:4, and a primer having a sequence comprising SEQ ID NO:5. Likewise, the composition can further comprise an amplification control nucleic acid and at least one oligonucleotide primer specific for the amplification control nucleic acid. It also can further comprising Taq polymerase and/or a dye that can specifically detect double stranded DNA, such as SYBR® Green I (Molecular Probes, Eugene, Oreg.).

In a fourth aspect, the invention provides kits for detecting Mycoplasma and/or Acholeplasma species in a sample. In its most basic form, the kit of the invention can comprise one or more nucleic acids or compositions according to the invention. The kits can comprise the components in a single package or in more than one package within the same kit. Where more than one package is included within a kit, each package can independently contain a single component or multiple components, in any suitable combination. As used herein, a combination of two or more packages or containers in a single kit is referred to as “in packaged combination”. The kits and containers within the kits can be fabricated with any known material. For example, the kits themselves can be made of a plastic material or cardboard. The containers that hold the components can be, for example, a plastic material or glass. Different containers within one kit can be made of different materials. In embodiments, the kit can contain another kit within it. For example, the kit of the invention can comprise a kit for purifying nucleic acids.

In general, the kits can comprise, in a single package or in packaged combination, two or more oligonucleotide primers, reagents and/or other components for performing the methods of the invention, nucleic acid templates for use as positive controls or specificity controls, or combinations of two or more of these.

In embodiments, the kit according to the invention comprises at least two primers, where each of the primers has a sequence comprising SEQ ID NO:1, SEQ ID NO:2, SEQ ID NO:3, SEQ ID NO:4, or SEQ ID NO:5, where each primer present in the kit has a sequence that differs from each other primer sequence in the kit. In embodiments, the kit comprises two primers, one comprising the sequence of SEQ ID NO:1 and the other comprising the sequence of SEQ ID NO:2. In other embodiments, the kit comprises three primers, one comprising the sequence of SEQ ID NO:1, the second comprising the sequence of SEQ ID NO:2, and the third primer comprising the sequence of SEQ ID NO:3, SEQ ID NO:4, or SEQ ID NO:5. In an exemplary embodiment, the kit comprises a primer comprising the sequence of SEQ ID NO:1, a primer comprising the sequence of SEQ ID NO:2, a primer comprising the sequence of SEQ ID NO:3, and a primer comprising the sequence of SEQ ID NO:4. In yet another exemplary embodiment, the kit comprises a primer comprising the sequence of SEQ ID NO:1, a primer comprising the sequence of SEQ ID NO:2, a primer comprising the sequence of SEQ ID NO:3, a primer comprising the sequence of SEQ ID NO:4, and a primer comprising the sequence of SEQ ID NO:5. In certain embodiments, all of the primers provided in the kit are provided in a single container, whereas in other embodiments, they are provided in at least two separate containers, alone or in combination with one or more other primer. In a specific embodiment, the kit can comprise a set of two or more, and preferably four or five, primers as described herein that recognize and amplify a 16S rRNA gene sequence from at least eight Mycoplasma/Acholeplasma species.

Accordingly, in embodiments, the invention provides a kit comprising at least one container holding a composition comprising at least one oligonucleotide primer, each of these primers having a sequence comprising the sequence of SEQ ID NO:1, SEQ ID NO:2, SEQ ID NO:3, SEQ ID NO:4, or SEQ ID NO:5. For example, the kit can comprise a container containing a primer having a sequence comprising SEQ ID NO:1, and a container containing a primer having a sequence comprising SEQ ID NO:2. Likewise, other containers can be provided that contain a primer having a sequence comprising SEQ ID NO:3, a primer having a sequence comprising SEQ ID NO:4, and a primer having a sequence comprising SEQ ID NO:5. Alternatively, two or more primers can be contained in one container, the invention not being limited by any particular combination of primers in each container.

The kit of the invention can comprise primers for the amplification control (AC). In embodiments, the AC primers are contained in a container separate from the other components of the kit. In other embodiments, the AC primers are contained in the same container as the Mycoplasma/Acholeplasma primers. In embodiments, the AC primers are contained in the same container as the AC template nucleic acid. In one embodiment, the AC primers, the AC template nucleic acid, and at least one Mycoplasma/Acholeplasma primer are contained in the same container. In exemplary embodiments, the AC primers individually comprise the sequences of SEQ ID NO:7 and SEQ ID NO:12. In other exemplary embodiments, the AC primers individually comprise the sequences of SEQ ID NO:6 and SEQ ID NO:8. In yet other exemplary embodiments, the AC primers individually comprise the sequences of SEQ ID NO:13 and SEQ ID NO:14.

The kit of the invention can comprise purified Mycoplasma and/or Acholeplasma nucleic acids. These nucleic acids can be genomic or sub-genomic nucleic acids. In exemplary embodiments, the kits comprise a container containing purified Mycoplasma and/or Acholeplasma gDNA. In embodiments, a known amount of Mycoplasma gDNA is contained in a single container within the kit. In embodiments, a known amount of Acholeplasma gDNA is contained in a single container within the kit. In certain cases, two containers, each containing one or the other of Mycoplasma or Acholeplasma gDNA, are included in the kit. In certain other cases, a Mycoplasma gDNA and an Acholeplasma gDNA are included in a single container within the kit. In embodiments, M. orale and A. laidlawii gDNA are provided in the kit, either in separate containers or together in a single container.

As mentioned above, the kit can comprise AC target nucleic acid. The AC target can be contained in a container separate from the other components of the kit, or in combination with one or more components. In embodiments, the AC target is contained in the same container as the AC primers. In embodiments, the AC target is contained in the same container as one or more of the Mycoplasma primers of the invention. The AC target nucleic acid can be any nucleic acid that comprises a sequence that can be amplified under the conditions used to amplify the target Mycoplasma/Acholeplasma sequence. Thus, it can be genomic or sub-genomic DNA. In exemplary embodiments, the AC target nucleic acid comprises mouse muscle nicotinic acetylcholine gamma receptor sequences. In other exemplary embodiments, the AC comprises human β-actin sequences.

The kit of the invention can comprise one or more components useful for amplifying the Mycoplasma/Acholeplasma target sequences. In embodiments, some or all of the reagents and supplies necessary for performing PCR are included in the kit. In exemplary embodiments, some or all reagents and supplies for performing QPCR are included in the kit. Non-limiting examples of reagents are buffers (e.g., a buffer containing Tris®, HEPES® and the like), salts, and a template-dependent nucleic acid extending enzyme (such as a thermostable enzyme, such as Taq polymerase), a buffer suitable for activity of the enzyme, and additional reagents needed by the enzyme, such as dNTPs, dUTP, and/or a UDG enzyme. In embodiments, the kit comprises Brilliant® SYBR® Green QPCR Master Mix (Catalog # 600548, Stratagene, La Jolla, Calif.). A non-limiting example of supplies is reaction vessels (e.g., microfuge tubes).

The kit can comprise at least one dye for detecting nucleic acids, including, but not limited to, dsDNA. In embodiments, the kit comprises a sequence-non-specific dye that detects dsDNA, such as SYBR® Green dye (Molecular Probes, Eugene, Oreg.). The dye is preferably contained alone in a container. In embodiments, the dye is provided as a concentrated stock solution, for example, as a 50× solution. In embodiments, the kit comprises a passive reference dye. In these embodiments, the passive reference dye can be included in the kit alone in a separate container. The passive reference dye can be provided as a concentrated stock solution, for example, as a 1 mM stock solution. A non-exclusive exemplary passive reference dye is ROX dye. In embodiments, the kit contains either a DNA-detecting dye or a passive reference dye. In other embodiments, the kit contains both a DNA-detecting dye and a passive reference dye.

The kit can also comprise one or more components useful for purifying nucleic acids. In embodiments, these components are particularly suited for purifying Mycoplasma/Acholeplasma nucleic acids from eukaryotic cell cultures. The components can be, among other things, reagents and supplies that can be used to purify nucleic acids. Non-limiting examples of such reagents and supplies include, but are not limited to, a DNA binding solution, a wash buffer, and containers, such as microfuge tubes, for collection of binding solutions, wash buffers, and purified nucleic acids. The components can also contain a resin, gel, or other substance that is useful for purifying nucleic acids. In embodiments, the kit comprises the components of the StrataPrep® PCR Purification Kit (Catalog # 400771, Stratagene, La Jolla, Calif.).

Thus, in embodiments, the kit according to the invention can comprise in packaged combination: 1) a primer set that comprises five Mycoplasma primers, one each comprising the sequence of SEQ ID NO:1, SEQ ID NO:2, SEQ ID NO:3, SEQ ID NO:4, and SEQ ID NO:5; 2) Mycoplasma orale gDNA; 3) Acholeplasma laidlawii gDNA; 4) a primer set for an AC that comprises two AC primers, one each comprising the sequence of SEQ ID NO:7 and SEQ ID NO:12; 5) an AC template that comprises genomic or sub-genomic sequences of the mouse muscle nicotinic acetylcholine receptor gamma-subunit that contain the sequences complementary to SEQ ID NO:7 and SEQ ID NO:12; 6) a Brilliant® SYBR® Green QPCR Master Mix; 7) a ROX dye solution; and 8) all of the reagents and supplies contained in Stratagene's StrataPrep® PCR Purification Kit.

### EXAMPLES

Various embodiments of the invention will now be described by way of a number of examples. The examples are presented solely to further describe certain embodiments of the invention, and are not to be construed as limiting the invention in any way.

### Example 1

### Extraction and Purification of Nucleic Acids from Cell Culture Supernatants

Cell cultures to be assayed for infection by Mycoplasma/Acholeplasma were grown to near confluence. One hundred microliters (100 μl) of the medium was transferred from the cell culture to a microcentrifuge tube. The tube was tightly closed to prevent opening during subsequent steps, particularly the heating step.

The microcentrifuge tube was incubated in a water bath at 95-100° C. for 5 minutes, then centrifuged for 30-60 seconds at top speed in a microcentrifuge. The supernatant (approx. 100 μl) was transferred to a new microcentrifuge tube.

One hundred microliters of the DNA-binding solution provided with the StrataPrep® PCR Purification Kit (Catalog # 400771, Stratagene, La Jolla, Calif.) and 200 μl of 70% (v/v) ethanol were added to the supernatant, and the combination was mixed well. StrataPrep Kit #400711 was used, except the spin cup in the kit were replaced by Stratagene Catalog #4151 spin cups.

Using a pipette, the mixture was transferred to a seated StrataPrep microspin cup in a 2 ml. receptacle tube, and sealed, according to the manufacturer's directions. The tube was then spun in a microcentrifuge at maximum speed for 30 seconds. The solution in the receptacle tube was discarded.

A wash buffer was prepared by adding 40 ml. of 100% ethanol to the bottle of 5× wash buffer supplied with the StrataPrep kit. Then, 750 μl of the wash buffer was added to the microspin cup, and the cap of the receptacle tube was snapped onto the top of the microspin cup. The tube was then spun in a microcentrifuge at maximum speed for 30 seconds.

The solution in the receptacle tube was discarded, and the capped microspin cup was placed back in the 2 ml. receptacle tube. The tube was spun in a microcentrifuge at maximum speed for 30 seconds to remove any residual wash buffer, then the microspin cup was transferred to a fresh 2 ml. microcentrifuge tube.

Fifty microliters of elution buffer (5 mM Tris (pH 8), 0.1 mM EDTA) was added directly onto the top of the fiber matrix at the bottom of the microspin cup, and the cap of the receptacle tube was snapped onto the microspin cup. The tube was incubated at room temperature for 5 minutes, then spun in a microcentrifuge at maximum speed for 30 seconds.

The purified template in elution buffer was stored in the receptacle tube.

### Example 2

### Extraction and Purification of Nucleic Acids from Cell Pellets

As discussed above, cell culture supernatants might contain media components that inhibit PCR amplification (e.g., fetal calf serum, metabolic products, antibiotics). This extraction protocol was used when it was desirable to minimize or eliminate such inhibitory components. It has been found that this protocol provides cell-equivalent standardization and a more sensitive detection limit for cell lines whose growth is inhibited by Mycoplasma. This protocol may be used as an alternative to testing cell culture supernatants that may be inhibitory to PCR.

Cell cultures to be assayed for infection by Mycoplasma/Acholeplasma were grown to near confluence. The cells were harvested by scraping the cells from the plate without prior addition of trypsin. One ml. of scraped adherent cells (100,000 or more cells) were transferred to a microcentrifuge tube and spun at top speed for 10-15 seconds. The supernatant was carefully decanted.

To wash the cells, the cells were resuspended in 1 ml. of sterile Dulbecco's phosphate-buffered saline (PBS), and the tube spun in a microcentrifuge for 10-15 seconds. The supernatant was carefully decanted, and the wash procedure was repeated. Alternatively, in some experiments, the cells were washed with unused culture medium.

The cells were resuspended in 1 ml. PBS, and counted under a microscope. One hundred thousand (100,000) cells were transferred to a fresh microcentrifuge tube and spun for 10-15 seconds. The supernatant was carefully aspirated with a micropipette, then 100 μl of sterile, UV-irradiated water was added to the cell pellet.

The cell suspension was incubated in a water bath at 95-100° C. for 10 minutes, then centrifuged for 30-60 seconds at top speed in a microcentrifuge. The supernatant (approx. 100 μl) was transferred to a new microcentrifuge tube.

One hundred microliters of the DNA-binding solution provided with the StrataPrep® PCR Purification Kit (Catalog # 400771, Stratagene, La Jolla, Calif.) and 200 μl of 70% (v/v) ethanol were added to the supernatant, and the combination was mixed well.

Using a pipette, the mixture was transferred to a seated StrataPrep microspin cup in a 2 ml. receptacle tube, and sealed, according to the manufacturer's directions. The tube was then spun in a microcentrifuge at maximum speed for 30 seconds. The solution in the receptacle tube was discarded.

A wash buffer was prepared by adding 40 ml. of 100% ethanol to the bottle of 5× wash buffer supplied with the StrataPrep kit. Then, 750 μl of the wash buffer was added to the microspin cup, and the cap of the receptacle tube was snapped onto the top of the microspin cup. The tube was then spun in a microcentrifuge at maximum speed for 30 seconds.

The solution in the receptacle tube was discarded, and the capped microspin cup was placed back in the 2 ml. receptacle tube. The tube was spun in a microcentrifuge at maximum speed for 30 seconds to remove any residual wash buffer, then the microspin cup was transferred to a fresh 2 ml. microcentrifuge tube.

Fifty microliters of elution buffer (5 mM Tris (pH 8), 0.1 mM EDTA) was added directly onto the top of the fiber matrix at the bottom of the microspin cup, and the cap of the receptacle tube was snapped onto the microspin cup. The tube was incubated at room temperature for 5 minutes, then spun in a microcentrifuge at maximum speed for 30 seconds.

The purified template in elution buffer was stored in the receptacle tube.

### Example 3

### Detection of Eight Different Species of Mycoplasma with One Primer Set

Eight Mycoplasma or Acholeplasma bacterial strains were obtained as lyophilized preparations from the American Type Culture Collection (ATCC, Manassas, Va.) as follows: Acholeplasma laidlawii (ATCC#23206), Mycoplasma orale (ATCC#29802), M. hominis (ATCC#23114), M. fermentans (ATCC# 19989), M. hyorhinis (ATCC#17981), M. pirum (ATCC#25960), M. salivariuni (ATCC#23064), M. arginini (ATCC#23243). E. coli (ATCC#l 0798) was also obtained for use in determining specificity of the primers. The Mycoplasma/Acholeplasma primer set described in Table 1 was tested for the detection of each of these eight different species of Mycoplasma and E. coli.

Crude DNA isolation was performed by resuspending lyophilized Mycoplasma bacterial cells in 1 ml of sterile double-deionized (ddI) water, followed by thorough mixing with a aerosol-filtered pipette tip. Resuspension in water ensured hydrolysis of the cells and release of the bacterial DNA. The cell solution was stored at −20° C. or immediately purified with a StrataPrep® PCR Purification Kit (Catalog # 400771, Stratagene, La Jolla, Calif.) according to the manufacturer's instructions. 1×10⁶ copies of each species' genome were used to determine whether the primer set of the present invention (SEQ ID NO:1-5, Table 1) were suitable for amplification/detection of Mycoplasma/Acholeplasma by QPCR using the methods of the present invention.

The set of reactions listed in Table 3 were prepared in duplicate. These include three types of control reactions: 1) an amplification control (AC) for each cell culture extract; 2) positive control reaction(s) containing Mycoplasma positive control template DNA, and 3) a negative (no template) control.

Reagent mixture for each tube:
17.25 μl Nuclease-free PCR-grade H₂O
25 μl of 2× Brilliant® SYBR® Green QPCR master mix
1 μl of Mycoplasma primer mix
0.75 μl of diluted reference dye (optional)

The Brilliant® SYBR® Green QPCR Master Mix (2×) contains SureStart® Taq polymerase (Catalog # 600280, Stratagene, La Jolla, Calif.). For PCR mixtures containing the Brilliant® Master Mix PCR enzymes, a 10-minute pre-incubation was included to activate the SureStart® polymerase component.

TABLE 3
QPCR Reaction Mixtures For Detection of Mycoplasma Species

| | Reaction Types | | | |
|---|---|---|---|---|
| | Cell | Amplification- | Mycoplasma- | Nega- |
| REACTION | Culture | Positive Controls | Positive | tive |
| COMPONENTS | Tests | (for each extract) | Control | Control |
| PCR Template | Cell | Cell extract + AC | M. orale or | None |
| | extract | | A. laidlawii | |
| | | | Positive | |
| | | | Control | |
| | | | Template | |
| Reagent Mixture | 44 μl | 44 μl | 44 μl | 44 μl |
| Test Cell Extract | 5 μl | 5 μl | — | — |
| Amplification | — | 1 μl | — | — |
| Control Solution | | | | |
| M. orale or | — | — | 5 μl | — |
| A. laidlawii | | | | |
| Positive Control | | | | |
| Template | | | | |
| H₂O | 1 μl | — | 1 μl | 6 μl |
| Final reaction | 50 μl | 50 μl | 50 μl | 50 μl |
| volume | | | | |

Five microliters (5 μl; 1×10⁶ copies) of the corresponding purified test culture template plus 1 μl of PCR-grade H₂O were added to the cell culture test reaction tubes. To the amplification positive control reaction tubes, 5 μl of the corresponding purified test culture template plus I II of amplification control template were added. To the Mycoplasma positive control reaction tubes, 5 μl of M. orale or A. laidlawii positive control template plus 1 μl of PCR-grade H₂O were added. To the negative control reaction tubes, 6 μl PCR-grade H₂O (or negative extract) were added.

The amount of DNA tested in the initial PCR (10⁶ copies) is reasonably comparable to the amount of Mycoplasma DNA that would be found in a contaminated culture (10⁷-10⁸ cells/ml of culture, 5 μl of extracted supernatant added to the PCR=5×10⁴−5×10⁵ input copies) (McGarrity, G. J. and H. Kotani, 1985).

Additional reactions can be performed, in which UDG is included. In these reactions, the mixtures are pre-incubated at 37° C. for 10 minutes for optimal hydrolysis of dUTP-containing DNA. Then the mixtures are incubated at 94° C. for 10 minutes to inactivate UDG.

TABLE 4
QPCR reaction cycling conditions.

| Cycles | Duration of cycle | Temperature |
|---|---|---|
| 1 | 10 minutes | 95° C. |
| 40 | 15 seconds | 95° C. |
| | 1.0 minute | 60° C. |
| | 30 seconds | 79° C. |

An Mx3000P® thermocycler (Stratagene, La Jolla, Calif.) was used to perform QPCR on the samples. The thermocycler was set to detect and report fluorescence during the annealing (60° C.) and extension (79° C.) cycles.

The results of the QPCR reactions are presented in FIG. 3. As can be seen in the Figure, all eight Mycoplasma/Acholeplasma species tested were detected using the primer set and method of the present invention.

### Example 4

### Determination of Dissociation Temperature of Mycoplasma PCR Products

The PCR products resulting from Example 3 were incubated at 95° C. for 1 minute to denature the products. Then, the thermocycler was ramped down to 55° C. The temperature was then ramped back up from 55° C. to 95° C. at a rate of 0.2° C. per second. Fluorescence data was continuously collected during the ramp up period.

FIG. 4A shows a dissociation curve of the 8 species tested (listed in FIG. 4B). The curve shows that the PCR products of all 8 species have a dissociation, or melting, temperature (Tm) of about 82° C. This property is reproducible and characteristic of the amplified Mycoplasma 16S rRNA sequence. Owing to these facts, it is used in embodiments of the present invention to identify the target PCR product, and to differentiate the target product from non-target PCR products that might be present in the PCR reaction mixture.

### Example 5

### Determination of Specificity of Mycoplasma Primers With Respect to Eukaryotes

To determine whether certain Mycoplasma primers of the invention specifically amplified Mycoplasma/Acholeplasma 16S rRNA sequences, but not sequences from human, mouse, or rat genomic DNA, PCR reactions were run with primers comprising SEQ ID NO:1-5 and nucleic acid templates from each of those organisms.

Amplification was performed using the Brilliant® SYBR® Green Master Mix with primers specific for the Mycoplasma 16S rRNA (200 nM), mouse muscle nicotinic acetylcholine receptor gamma-subunit Amplification Control (AC) (50 nM), or human β-actin (300 nM, Stratagene #302010-14). Primers specific for the human β-actin were designed based on the known sequence of the β-actin gene to provide an amplification product of a known size. Each primer set was mixed with genomic DNA from either Mycoplasma orale (10⁵ input copies; “My”), mouse (100 ng; “M”), human (100 ng; #1193-1; “H”), rat (100 ng; “R”), plasmid pBMG419 (10⁶ copies; containing the mouse muscle nicotinic acetylcholine receptor gamma-subunit DNA; “P”), or no DNA (no-template control, “−”), and PCR run according to standard protocols.

The PCR reaction results were analyzed by gel electrophoresis for amplification of the templates. For gel electrophoresis, 10-μl samples of 50-μl amplification reactions were fractionated on a 2% agarose gel containing 0.125 μg/ml ethidium bromide (SeaKem L E, BioWhittaker Molecular Applications #50004). While 2% gels produce the greatest resolution between AC and target bands, 1% gels produce acceptable results (data not shown). Gels were run at 120V in 1× TAE for about 55 minutes. Molecular weight marker (Stratagene #201101) were used to estimate molecular weight. Gels were imaged using the Eagle Eye II Still Video System (Stratagene). The far left and right lanes contain size makers (φX 174 HaeIII, Stratagene #201101). Genomic DNA was obtained from the Stratagene Production group at BioCrest, although any suitably pure DNA could be used.

FIG. 5 shows the results of gel readout. It shows that a Mycoplasma 16S primer set according to the invention amplifies Mycoplasma sequences, as expected (“My” lane). The figure also shows that the tested primer set does not amplify a product from human, rat, or mouse gDNA templates (“H”, “M”, and “R” lanes), indicating that it does not produce false-posifive results due to the presence of DNA from cultured cells. The amplification control primer set only yields an amplification product with its matched target: plasmid pBMG419 (lane “P”). Note that the control β-actin primers cross-react between human, rat, and mouse species (“H”, “M”, “R” lanes in β-actin section of gel). This control shows that the genomic DNA is readily amplifiable and does not inhibit PCR.

Table 5 summarizes the results of the QPCR reactions for which gel readout was performed and presented in FIG. 5. Table 5 shows that the Mycoplasma primer set according to the present invention specifically amplified Mycoplasma orale gDNA, resulting in a Ct of 21. 1, the AC primer set specifically amplified its target sequences on plasmid pBMG419, and the human β-actin primer set hybridized only to β-actin sequences, but not Mycoplasma or mouse muscle nicotinic acetylcholine gamma subunit sequences. Taken together, these results confirm that the Mycoplasma primer set is specific for Mycoplasma sequences, that the AC primer set is specific for its target sequences, and that the QPCR reaction successfully ran as designed. N/A indicates that data was not collected for that combination.

TABLE 5
Test of Mycoplasma and AC Primers with Human, Mouse, and Rat gDNA

| | | | | | pBMG419 |
|---|---|---|---|---|---|
| | Human | Mouse | Rat | Mycoplasma | DNA |
| Mycoplasma | No Ct | No Ct | No Ct | 21.1 (10⁵ copies | N/A |
| primers | | | | Mycoplasma | |
| | | | | DNA) | |
| AC Primers | No Ct | No Ct | No Ct | N/A | 17.5 (10⁶ |
| | | | | | copies |
| | | | | | of pDNA) |
| β-actin | 17.7 | 21.2 | 19.2 | N/A | N/A |
| Primers | | | | | |

### Example 6

### Determination of Specificity of Primers With Respect to E. coli

To confirm the results obtained in Example 6, QPCR was performed on E. coli DNA (see above for source, purification, and amplification conditions). Four different amplification reactions were run: 1) amplification of Mycoplasma gDNA sequences using purified Mycoplasma DNA as template and a Mycoplasma primer set; 2) amplification of E. coli gDNA sequences using purified E. coli DNA as template and primers specific for E. coli rDNA sequences; 3) amplification of E. coli gDNA with Mycoplasma specific primers, and 4) a negative control (no DNA added as template) to determine if nucleic acids that could be amplified by the Mycoplasma primer set were present in the amplification mixture without added DNA.

FIG. 6 shows the QPCR readout for the reactions described above. As can be seen from FIG. 6, the Mycoplasma primer set tested specifically amplified a sequence present in Mycoplasma gDNA, but did not amplify any sequences in E. coli gDNA. Likewise, the primer set designed to detect E. coli detected E. coli, confirming that the E. coli template used was amplifiable. Finally, the figure shows that there was no amplification products from the Mycoplasma primer set tested in the absence of added Mycoplasma DNA, indicating that the amplification curve presented for Mycoplasma amplification is, in fact, due to amplification of Mycoplasma DNA and not contaminating DNA present in one or more of the QPCR mix components.

In performing the experiments reported in the figure, E. coli DNA amounts from 1 ng to 10 pg were tested. No Ct was observed up to 40 cycles for any of these concentrations when the Mycoplasma primer set was used, showing that the primer set according to the present invention is specific for Mycoplasma/Acholeplasma over E. coli.

### Example 7

### Detection of Mycoplasma In A Two-Tube Format of the Invention

As discussed above, one optional embodiment of the present methods comprises running a positive control in the form of an amplification control (AC). In general, the amplification control is designed to confirm the absence of PCR inhibitors in culture samples being tested for Mycoplasma infection. The AC comprises a template that does not show cross-hybridization with primers of the Mycoplasma primer set. The AC also comprises at least two primers that are specific for the AC template (i.e., amplify the AC template but not any Mycoplasma nucleic acids that might be present).

In separate tubes, 1×10⁵ genome copies of eight Mycoplasma/Acholeplasma species (see Example 3, above) were subjected to QPCR in accordance with the protocols given above. Four duplicates, each in a separate tube, of an AC was run. For the AC reactions, 1,000 copies of the mouse muscle nicotinic acetylcholine receptor gamma-subunit was used as a template. All eight Mycoplasma species, and the one AC, produced a PCR product (QPCR fluorescence or gel electrophoresis data not shown). The resulting PCR products were used to determine the Tm for each, in accordance with the protocol discussed in Example 4, above. The results are presented in FIG. 7A. FIG. 7B lists the Tm for each species.

The data presented in FIG. 7 show once again that the specifically produced PCR product from Mycoplasma has a Tm of about 82° C. throughout the eight Mycoplasma/Acholeplasma species tested. The figure also shows that the method of the invention successfully amplified all eight Mycoplasma/Acholeplasma species, and amplified the AC. Finally, the figure shows that the Tm for the AC used in the experiment is detectably different than the Tm for the Mycoplasma/Acholeplasma species. Thus, the figure shows that the Tm of the Mycoplasma/Acholeplasma and AC can be used to differentiate the two from each other. That is, the figure shows that an analysis of Tm can tell one whether Mycoplasma/Acholeplasma nucleic acid has been amplified, the AC has been amplified, or both.

### Example 8

### Detection of Mycoplasma In A Single-Tube Format of the Invention

The results obtained in Example 7 indicate that the PCR product from mouse muscle nicotinic acetylcholine receptor gamma-subunit, when used as the AC, can be differentiated from the PCR product from Mycoplasma/Acholeplasma based on Tm alone. That is, the amplification product of the AC can be differentiated from the amplification product of the sample target without the need to 1) run a gel to determine the presence and size of each band, and 2) run two separate reactions in two separate tubes. To confirm that these conclusions are valid, eight QPCR reactions were run. Each reaction contained gDNA from one Mycoplasma species plus the AC such that the eight reactions separately contained the eight Mycoplasma species. The mouse muscle nicotinic acetylcholine receptor gamma-subunit gene (with the corresponding primer set) was used as the AC, and was present in the same reaction mix and tube as each of the eight Mycoplasma species. The results are presented in FIG. 8.

FIG. 8A shows that all eight Mycoplasma/Acholeplasma species and the AC can be detected when each Mycoplasma species and the AC are amplified in eight single QPCR reaction mixtures. Furthermore, the figure shows that the two PCR products can be differentiated from each other by Tm (FIGS. 8A and 8B).

This result points out one of the advantages the present invention provides over other Mycoplasma detection assays that are currently known. More specifically, the results presented in FIG. 8 show that a QPCR reaction for detection of Mycoplasma/Acholeplasma can be run in which two templates are simultaneously detected. In the reaction, two sets of primers are simultaneously used, each specific for a different target nucleic acid. Yet, although two distinct targets can be detected, only one dye is necessary and no gel electrophoresis is required to identify and differentiate the two distinct PCR products.

### Example 9

### Effect of Concentrations of Mycoplasma Target and AC in the Single-Tube Assay of the Invention

The effect of varying concentrations of Mycoplasma target on a constant concentration of AC on detection of Mycoplasma target was determined. In this experiment, 1000 copies of AC and 50 nM of AC primers were mixed with varying amounts of M. orale gDNA to determine whether the relative abundances affected the production of PCR product. FIG. 9(A-C) shows the effect of varying concentrations of M. oral gDNA. FIG. 9A shows a dissociation curve when 1,000 copies of M. orale gDNA are included in the reaction mixture. FIG. 9B shows a dissociation curve when 100 copies of M. orale gDNA are included in the reaction mixture. FIG. 9C shows a dissociation curve when 10 copies of M. orale gDNA are included in the reaction mixture.

FIG. 9 shows that as few as 10 copies of M. orale can be detected in this assay using Tm. It also shows that use of 1,000 copies of the AC and 50 nM of AC primer provides good results for detecting Mycoplasma nucleic acids in samples. Finally, using 1,000 AC copies and 50 nM of AC primers appears to reduce the formation of primer-dimers (PD) as well (data not shown).

### Example 10

### Sensitivity of a Mycoplasma Primer Set According to the Invention

The performance of the methods of the invention at various DNA copy numbers was examined. Based upon rough estimates of the size of Mycoplasma genomes (Neimark & Lange, 1990), the concentrations of the crude genomic DNA preparations for each species isolated above were calculated in copies/μl and normalized for each species. The DNAs were titrated into reactions containing the 16S rRNA Mycoplasma primer mix.

Six QPCR reactions were run in duplicate as described above, with the following variation. One of each of the six reaction tubes contained the following amounts of M. orale gDNA: zero copies, 10 copies, 100 copies, 1,000 copies, 10,000 copies, and 100,000 copies. Each reaction was performed in duplicate. The results of the QPCR reactions are shown in FIG. 10. FIG. 10 shows, among other things, the sensitivity of the present methods for detecting M. orale genomic DNA. More specifically, it shows that the methods of the present invention can detect as few as 10 copies of M. orale genomic DNA in a sample, such as a HeLa cell culture supernatant, in 35 cycles. It also shows that the methods are suitable for detecting as many as 100,000 copies, or more, of M. orale genomic DNA. Due to the high identity between the target regions for the primer set for Mycoplasma/Acholeplasma genomic DNA, it is evident that the present methods would provide similar results and sensitivity for other members of the Mycoplasma and Acholeplasma genera.

Table 6 shows the results of a separate assessment of the Mycoplasma primers of the invention. The table shows that, under optimized conditions, the present methods can detect a single Mycoplasma or Acholeplasma genome in a sample. To generate the data, ten-fold serial dilutions of purified Mycoplasma genomic DNA was made in 5T 0.1E buffer for the 8 different species of Mycoplasma. Each serial dilution was tested in triplicates on the Mx3000P real-time reader (Stratagene) using 1× Brilliant® SYBR® Green master mix, Mycoplasma primer set according to the invention, and ROX passive reference dye. The lowest concentration at which a target dependent amplification is observed is the limit of detection shown here.

TABLE 6
Detection Limits of Mycoplasma Species

| | Detection Limit |
|---|---|
| Species: | (copies of genome): |
| M. arginini | 100 |
| M. fermentans | 10 |
| M. hominis | 10 |
| M. hyorhinis | 10 |
| M. orale | 1 |
| M. pirum | 100 |
| M. salivarium | 100 |
| A. laidlawii | 1 |

### Example 11

### Comparison of the Present Methods With a Commercial Product

To determine the relative sensitivities of the present methods and those that can be practiced using a commercially available QPCR kit for detection of Mycoplasma, the VenorGeM® QP kit from Minerva was used to detect M. oral genome DNA. The kit was used according to the manufacturer's instructions. Ten, 100, 1,000, and 10,000 copies of M. orale gDNA was used as a template for QPCR amplification in the VenorGeM® QP kit. The results are presented in FIG. 11A, which shows that 38 cycles are required to detect 10 copies of M. orale gDNA, and 23 cycles are required to detect 10,000 copies. The figure also shows that the curves are generally flattened, with a tailing off of the increase in fluorescence occurring gradually and not until well past cycle 40. Further, the figure shows that total fluorescence intensity is in the 1,000-7,000 range.

FIG. 11B shows a standard curve created from the data obtained from FIG. 11A. The standard curve shows that the Minerva VenorGeM® QP kit provides substantial linearity from about 10 copies to about 100,000 copies of M. orale gDNA. The curve shows that the efficiency of amplification was 86.6%, and that the theoretical Ct for one genome is 41.2.

The methods of the present invention were performed using the same M. orale gDNA that was used to evaluate the sensitivity of the Minerva VenorGeM® QP kit. The results of the present methods are shown in FIG. 12A. FIG. 12A shows that the present methods permit one to detect 10 copies of M. orale gDNA in as few as 31 cycles, and that 10,000 copies can be detected in as few as 18 cycles. In contrast to the commercially available product tested above, the curves created by the present invention are nearly sigmoidal, indicating that the QPCR reactions are running to completion earlier than the kit tested above. Further, the figure shows that the total fluorescence intensity is in the 10,000-20,000 or higher range, indicating a very robust system.

FIG. 12B shows a standard curve created from the date obtained from FIG. 12A. The standard curve shows that the methods of the present invention provide essentially perfect linearity from about 10 copies to about 100,000 copies of Mycoplasma DNA. The curve also shows that the efficiency of amplification was 97.2%, and that the theoretical Ct for one genome is 34.

It is evident from this comparison that the present methods can be more sensitive than methods that are currently publicly available, and that they can reduce the time necessary to identify Mycoplasma infection of a sample by reducing the number of cycles necessary to detect the bacteria.

All patents, patent applications, and published references cited herein are hereby incorporated by reference in their entirety. While this invention has been particularly shown and described with references to various embodiments, it is to be understood that various changes can be made without departing from the scope of the invention.

### REFERENCES

The following references are cited herein, and are hereby incorporated in their entireties into this document by reference.
Altschul et al., Nucleic Acids Res. 25:3389-3402, 1997.
Barile, M. F. and S. Razin, The Mycoplasmas, New York, Academic Press: 425-474, 1979.
Blazek et al., J. Immunol. Meth. 131:203-212, 1990.
Corless, C. E., et al., “Contamination and Sensitivity Issues with Real-Time Universal 16S rRNA PCR”, J. Clin. Microb. 38(5):1747-1752, 2000.
DelGuidice, R. A. and H. E. Hopps, Mycoplasma infection of cell cultures, G. J. McGarrity, D. G. Murphy and W. W. Nichols, New York, Plenum Press: 57-69, 1978.
Dussurget and Roulland-Dussoix, Appl. Environ. Microbiol. 60:953-959, 1994.
Eastick et al., J. Clin. Pathol.: Mol. Pathol. 56:25-28, 2003.
Gingeras et al., Annales de Biologie Clinique, 48(7):498-501, 1990.
Guatelli et al., Proc. Natl. Acad. Sci. USA., 87:1874, 1990.
Harasawa et al., Res. Microbiol. 144:489-493, 1993.
Higuchi et al., Biotechnology (N Y) 11(9):1026-30, 1993.
Hopert et al., J. Immunol. Meth. 164:91-100, 1993.
Kong, F et al., “Species-specific PCR for identification of common contaminant mollicutes in cell culture”, Appl Environ Microbiol 67(7):3195-3200, 2001.
Loens et al., J. Clin. Microbiol. 40:339-1345, 2002.
McGarrity, G. J. and Kotani, The Mycoplasmas, New York, Academic Press, pp. 353-390, 1985.
McGarrity et al., In Vitro Cell. Dev. Biol. 22:301-304, 1986.
Neimark, H. C. and C. S. Lange, “Pulse-field electrophoresis indicates full-length Mycoplasma chromosomes range widely in size”, Nucleic Acids Res 18(18):5443-5448, 1990.
Tang, J. et al., “Primer mixture enhances PCR detection of Mycoplasma/Acholeplasma contaminants in cell cultures.” In Vitro Cell Dev Biol. 35(1):1-3, 1999.
Tatusova & Madden, FEMS Microbiol. Lett. 174:247-250, 1999.
Uphoff et al., Leukemia 6:335-341, 1992.
Uphoff et al., Leukemia 16:289-293, 2002.
van Kuppeveld, Appl. Environ. Microbiol. 58:2606-2615, 1992.
van Kuppeveld, Appl. Environ. Microbiol. 60:149-152, 1994.
Walker, Nucleic Acids Res. 22:2670-2677, 1994.
Wirth et al., Cytotechnology 16:67-77, 1994.
Yoshida et al., J. Clin. Microbiol. 40:1451-1455, 2002.

1. An oligonucleotide primer having a sequence comprising the sequence of SEQ ID NO:1, SEQ ID NO:2, SEQ ID NO:3, SEQ ID NO:4, or SEQ ID NO:5.

2. A composition comprising at least one oligonucleotide primer, each of said at least one primer having a sequence comprising the sequence of SEQ ID NO:1, SEQ ID NO:2, SEQ ID NO:3, SEQ ID NO:4, or SEQ ID NO:5.

3. The composition of claim 2, comprising a primer having a sequence comprising SEQ ID NO:1 and a primer having a sequence comprising SEQ ID NO:2.

4. The composition of claim 3, further comprising a primer having a sequence comprising SEQ ID NO:3, a primer having a sequence comprising SEQ ID NO:4, and a primer having a sequence comprising SEQ ID NO:5.

5. The composition of claim 2, further comprising an amplification control nucleic acid and at least one oligonucleotide primer specific for said amplification control nucleic acid.

6. The composition of claim 2, further comprising Taq polymerase.

7. The composition of claim 2, further comprising a dye that can specifically detect double stranded DNA.

8. A kit comprising the composition of claim 2 in a first container.

9. The kit of claim 8, further comprising genomic or sub-genomic Mycoplasma or Acholeplasma nucleic acids in a second container.

10. The kit of claim 8, further comprising an amplification control nucleic acid, wherein said amplification control nucleic acid is present in said first container or in a second container.

11. The kit of claim 8, further comprising reagents and supplies for purification of nucleic acids.

12. The kit of claim 8, further comprising, in packaged combination, SYBR Green dye, an amplification control, a Mycoplasma control, an Acholeplasma control, a reference dye, a template dependent nucleic acid extending enzyme, and all reagents and supplies necessary to purify nucleic acids.

13. A method of acellular amplification of Mycoplasma or Acholeplasma nucleic acids, said method comprising
providing a sample suspected of containing a purified nucleic acid from Mycoplasma or Acholeplasma;
providing at least two oligonucleotide primers, each of said primers having a sequence comprising the sequence of SEQ ID NO:1, SEQ ID NO:2, SEQ ID NO:3, SEQ ID NO:4, or SEQ ID NO:5;
amplifying said purified Mycoplasma or Acholeplasma nucleic acid; and
detecting the product of the amplifying reaction.

14. The method of claim 13, wherein said detecting comprises detecting amplification in real-time, or gel electrophoresis of the amplification reaction followed by detecting amplification in real-time.

15. The method of claim 13, wherein said method is a method of quantitative PCR (QPCR).

16. The method of claim 13, wherein said extension product is detected by SYBR Green staining.

17. The method of claim 13, wherein the method detects Acholeplasma laidlawii, Mycoplasma arginini, M. fermentans, M. hominis, M. hyorhinis, M. orale, M. salivariutn, M. pirum, or a combination of two or more of these organisms.

18. The method of claim 12, further comprising
providing an amplification control nucleic acid and at least two primers that specifically hybridize to the amplification control;
amplifying the amplification control; and
detecting the product of the amplification control amplifying reaction.

19. The method of claim 18, wherein said amplifying and detecting of said purified Mycoplasma or Acholeplasma nucleic acid and said amplifying and detecting of said amplification control are performed in a different reaction tube.

20. The method of claim 18, wherein said amplifying and detecting of said purified Mycoplasma or Acholeplasma nucleic acid and said amplifying and detecting of said amplification control are performed in the same reaction tube.

21. The method of claim 20, wherein the method is a QPCR method, and wherein double-stranded nucleic acids are detected using SYBR Green dye.
